(19) United States
(12) Patent Application Publication
(10) Pub. No.: US 2025/0303183 A1
(43) Pub. Date: Oct. 2, 2025

(54) IMPLANTABLE MEDICAL DEVICE FEEDTHROUGH ASSEMBLY AND SUBSTRATES THEREFOR WITH FLUID CONTROL BED

(71) Applicant: Medtronic, Inc., Minneapolis, MN (US)

(72) Inventors: John C. Mertz, Lino Lakes, MN (US); Lea Ann Nygren, Bloomington, MN (US); Andrew J. Thom, Maple Grove, MN (US); Alan Shi, Plymouth, MN (US); Yangbin Chen, Lake Elmo, MN (US); Shawn C. Kelley, Shoreview, MN (US); Everett A. Kroll, Woodbury, MN (US); Thomas Wendell Hovind, Andover, MN (US); Eric M. Stetz, Lino Lakes, MN (US); Steven T. Deininger, Stillwater, MN (US); Bin Zhou, Woodbury, MN (US)

(21) Appl. No.: 19/094,221

(22) Filed: Mar. 28, 2025

Related U.S. Application Data
(60) Provisional application No. 63/571,502, filed on Mar. 29, 2024.

(51) Int. Cl. A61N 1/375

(52) U.S. Cl. CPC A61N 1/3754

(57) ABSTRACT

Feedthrough assemblies for implantable medical devices and substrates therefore are provided herein including feedthrough bores extending through the substrates from a first major surface to a second major surface and including at least one fluid control beds on the first major surface. The fluid control beds are configured to modify interactions between the fluid control bed and a fluid, as compared with interactions between the fluid and other portions of the substrate.

### CROSS-REFERENCE TO RELATED APPLICATIONS

This application claims the benefit of U.S. Provisional Patent Application Ser. No. 63/571,502, filed Mar. 29, 2024. Each of the applications and patents listed in this paragraph is incorporated herein by reference in its entirety.

### TECHNICAL FIELD

This disclosure generally relates to, among other things, feedthrough assemblies for use in implantable medical devices (IMDs) and, more specifically, feedthrough assemblies and substrates therefor with fluid control beds and methods of making the same.

### BACKGROUND

Various systems require electrical coupling between electrical devices disposed within a sealed enclosure or housing and devices or systems external to the enclosure. For example, IMDs (e.g., cardiac pacemakers, defibrillators, neurostimulators, drug pumps, etc.) typically include electronic circuitry and one or more power sources and require an enclosure or housing to contain and seal these elements within a patient's body. Many such IMDs include one or more feedthrough assemblies to provide electrical connections between the elements contained within the housing and components of the IMD external to the housing (e.g., one or more sensors, electrodes, lead wires mounted on an exterior surface of the housing, electrical contacts housed within a connector header mounted on the housing to provide coupling for one or more implantable leads, etc.). A feedthrough assembly may be described as an apparatus that provides electrical coupling between electrical devices disposed within a sealed enclosure or housing and devices or systems external to the enclosure in an electrically-insulated and hermetically-sealed manner.

Implantable medical devices generally use various compositions to bond feedthrough pins within a feedthrough ferrule, or feedthrough bore, to form a hermetic seal between an internal volume of the implantable medical device and an environment external to the implantable medical device, and to electrically insulate the pin from the ferrule. Potting material may be applied in and around the feedthrough bore to provide additional, or supplemental, electrical insulation, such as between the pin and the ferrule. Because internal components may commonly be sensitive to the aqueous environment of the patient's body and to electrical shorts, it is important to isolate sensitive internal components, both fluidly and electrically.

### SUMMARY

As described herein, feedthrough assemblies and substrates for use therewith may be provided with fluid control beds, which may abut at least one feedthrough bore and defining an area about the feedthrough bore to contain potting material. In some cases, potting material compositions (e.g., liquid curable potting adhesives) applied to the feedthrough cavities of a feedthrough assembly may form interconnects with adjacent feedthrough cavities. Interconnects may form, for example, due to suboptimal placement of the potting material or overapplication of the potting material. It will be understood in light of this disclosure that there is a need to prevent material interconnection between adjacent feedthrough cavities in feedthrough assemblies to improve insulation performance of the potting material compositions.

Furthermore, as implantable medical devices are capable of being made in smaller and smaller form factors, the available space for forming sealing and insulating (e.g., hermetic sealing, electrical insulating, etc.) may be likewise reduced, requiring greater sealing efficacy. On the other hand, the extent to which a device's form factor can be reduced in size may be limited by the space needed for forming seals, such that improving sealing efficacy may afford reduced form factor size. It will be understood in light of this disclosure that there is a need to improve sealing efficacy of feedthrough assemblies to thereby improve smaller implantable medical device form factors.

Fluid control beds provided herein may be described as defining an area on a substrate's surface (e.g., about, or at least partially surrounding one or more feedthrough bores) and configured to modify interactions between the fluid control bed and a fluid, as compared with interactions between the fluid and other portions of the substrate.

For example, a fluid control bed may be configured to control, manage, or contain a fluid (e.g., medical adhesives, potting materials, etc.) applied to the respective feedthrough cavity. As described herein, such fluid control beds may advantageously prevent potting material of one cavity from overflowing into an adjacent cavity, or may prevent potting material of one cavity from contacting potting material of an adjacent cavity. Accordingly, such fluid control beds may prevent interconnection (via overflown potting material) of feedthrough pins disposed in adjacent feedthrough cavities.

Additionally or alternatively, a fluid control bed may include a surface texture configured to modify interactions between the fluid control bed and a fluid, as compared with interactions between the fluid and other portions of the substrate. For example, a surface texture may be configured to control, manage, or contain a fluid (e.g., medical adhesives, potting materials, etc.), for example, by preferentially wicking the fluid. Such surface textures may advantageously prevent potting material of one cavity from overflowing into an adjacent cavity, or may prevent potting material of one cavity from contacting potting material of an adjacent cavity. As another example, a surface texture may be configured to increase surface area of the fluid control bed capable of interaction with a fluid (e.g., medical adhesives, potting materials, etc.), which may be referred to herein as a “bonding surface texture.” In particular such bonding surface textures may may advantageously increase the extent of interaction between a liquid adhesive (i.e., a fluid) and the fluid control bed (i.e., a surface), such as by increasing the surface area of the fluid control bed capable of interaction (e.g., interface) with the liquid adhesive to form bonds therebetween, increasing tortuosity of a bond interface between the liquid adhesive and the fluid control bed, and/or increasing an extent of mechanical interlock between the liquid adhesive and the fluid control bed. Bonding surface textures providing increased surface area, tortuosity, and/or mechanical interlock of the fluid control bed may advantageously improve the durability of bonds, such as between the first major surface and an adhesive.

Embodiments disclosed herein may include a substrate for a feedthrough assembly for an implantable medical device. The substrate includes a first major surface and an opposing second major surface. At least first and second feedthrough cavities are defined by the substrate, and each extends through the substrate from the first major surface to the second major surface. Each feedthrough cavity includes a respective feedthrough bore configured to receive a feedthrough pin. The substrate further includes at least first and second fluid control beds on the first major surface. The first fluid control bed forms a first area about the first feedthrough bore configured to preferentially contain, to the first fluid control bed, potting material applied to the first feedthrough bore. The second fluid control bed forms a second area about the second feedthrough bore configured to preferentially contain, to the second fluid control bed, potting material applied to the second feedthrough bore. The first and second fluid control beds are separated from one another.

Embodiments disclosed herein may further include a feedthrough assembly including the substrate and a first feedthrough pin disposed within the first feedthrough bore and configured to electrically connect a component in an internal volume of the implantable medical device with a component in communication with an environment external to the implantable medical device. The first feedthrough pin may include a pin fluid control exterior surface texture. The pin fluid control surface exterior texture may include a plurality of axial grooves to draw the potting material from the first feedthrough bore. The plurality of axial grooves may be disposed radially about the pin and may extend axially along the pin away from the first feedthrough bore. The pin fluid control exterior surface texture may further include a stop abutting the plurality of axial grooves and including at least one radial groove.

Embodiments disclosed herein may still further include an implantable medical device including a housing defining an internal volume and the feedthrough assembly. The feedthrough assembly may be fixed relative to the housing.

The first fluid control bed may define a fluid control channel at least partially surrounding the first feedthrough bore and forming part of the first feedthrough cavity. The first fluid control bed may optionally further define an outer channel at least partially surrounding the fluid control channel. The first fluid control bed may include a bed fluid control surface texture defining a plurality of grooves. The bed fluid control surface texture may be configured to preferentially wick a potting material compared to other portions of the first major surface. The plurality of grooves may include a plurality of concentric grooves at least partially surrounding the first feedthrough bore. The plurality of grooves may include a plurality of axial grooves substantially normal to and at least partially surrounding the first feedthrough bore. The plurality of grooves may include a first plurality of substantially parallel grooves and a second plurality of substantially parallel grooves substantially perpendicular to the first plurality of substantially parallel grooves. Each groove of the plurality of grooves may independently have a depth of between 10 micrometers (um) and 20 um, between 5 um and 40 um, or 15 um. The plurality of grooves may have a minimum-to-minimum period of between 10 um and 120 um, between 20 um and 100 um, or 20 um. The plurality of grooves may have a groove aspect ratio of between 1:1 and 1:5, between 1:2 and 1:4, or 1:2. The first fluid control bed may have a depth measured from the major surface of between 10 um and 20 um, between 5 um and 40 um, or 15 um.

Embodiments disclosed herein may yet further include a method for forming an implantable medical device feedthrough assembly, the method including providing a substrate having a first major surface and an opposing second major surface, and defining at least first and second feedthrough cavities each extending through the substrate from the first major surface to the second major surface, wherein each feedthrough cavity includes a respective feedthrough bore. The method further includes forming at least first and second fluid control beds on the first major surface. The first fluid control bed forms a first area about the first feedthrough bore configured to preferentially contain, to the first fluid control bed, potting material applied to the first feedthrough bore. The second fluid control bed forms a second area about the second feedthrough bore configured to preferentially contain, to the second fluid control bed, potting material applied to the second feedthrough bore. The method further includes bonding at least first and in second feedthrough pins within the respective first and second feedthrough bores using a potting material.

The forming at least first and second fluid control beds on the first major surface may include forming a fluid control channel abutting and at least partially surrounding the first feedthrough bore. The fluid control bed may optionally define an outer channel abutting and at least partially surrounding the fluid control channel. The forming at least first and second fluid control beds on the first major surface may include forming a bed fluid control surface texture comprising a plurality of grooves. The forming at least first and second fluid control beds on the first major surface may include bead blasting the first major surface, laser etching the first major surface, laser spot-welding the first major surface, machining the first major surface, micro-etching the first major surface, or a combination of two or more thereof.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

FIG. 1A is an illustrative implantable medical device implanted in a patient including a feedthrough assembly with a fluid control bed.

FIG. 1B is a schematic perspective view of the implantable medical device of FIG. 1A.

FIG. 1C is a schematic exploded view of the implantable medical device of FIG. 1A.

FIG. 2A is a cross-sectional side view of an illustrative feedthrough assembly including a fluid control bed with certain features omitted for clarity.

FIG. 2B is a cross-sectional side view of the illustrative feedthrough assembly of FIG. 2A including potting material, insulating plugs, and feedthrough pins.

FIG. 2C is a top schematic view of the illustrative feedthrough assembly of FIG. 2B.

FIGS. 3A and 3B show the feedthrough assembly of FIGS. 2A-2C with fluid control beds having an outer channel abutting and surrounding the fluid control channels. FIG. 3A is a cross-sectional side view. FIG. 3B is a top schematic view.

FIG. 4 is a top view of an illustrative feedthrough assembly including auxiliary channels.

FIGS. 5A and 5B show an illustrative feedthrough assembly with fluid control beds having bed fluid control surface textures. FIG. 5A is a cross-sectional side view. FIG. 5B is a top schematic view.

FIGS. 6A-6H show substrates for forming the feedthrough assembly of FIGS. 5A and 5B including additional or alternative illustrative bed fluid control surface textures.

FIG. 7 shows an illustrative method of forming a feedthrough assembly.

FIGS. 8A and 8B show an illustrative feedthrough assembly including a fluid control bed with a bonding surface texture in cross-sectional side view (8A) and a schematic top view with certain features omitted for clarity (8B).

FIG. 9 shows an illustrative method of forming a feedthrough assembly, such as the feedthrough assembly of FIGS. 8A and 8B.

FIGS. 10A-17D show scanning electron microscopy (SEM) images of titanium coupons textured by femtosecond laser machining including one-pass lines (10A-10D), two-pass lines (11A-11D), five-pass lines (12A-12D), one-pass grids (13A-13D), two-pass grids (14A-14D), five-pass grids (15A-15D), dimples spaced on-center (16A-16D), and dimples staggered (17A-17D) at various magnifications.

The figures are rendered primarily for clarity and, as a result, are not necessarily drawn to scale. Moreover, various structure/components may be shown diagrammatically or removed from some of or all the views to better illustrate aspects of the depicted embodiments, or where inclusion of such structure/components is not necessary to an understanding of the various exemplary embodiments described herein. The lack of illustration/description of such structures/components in a particular figure is, however, not to be interpreted as limiting the scope of the various embodiments in any way.

### DETAILED DESCRIPTION

All scientific and technical terms used herein have meanings commonly used in the art unless otherwise specified. The definitions provided herein are to facilitate understanding of certain terms used frequently herein and are not meant to limit the scope of the present disclosure.

Unless otherwise indicated, the terms “polymer”, “polymerized monomers”, and “polymeric material” include, but are not limited to, organic homopolymers, copolymers, such as for example, block, graft, random and alternating copolymers, terpolymers, etc., and blends and modifications thereof. Furthermore, unless otherwise specifically limited, the term “polymer” shall include all possible geometrical configurations of the material. These configurations include, but are not limited to, isotactic, syndiotactic, and atactic symmetries.

In this disclosure, all numbers are assumed to be modified by the term “about,” which encompasses the term “exactly.” As used herein in connection with a measured quantity, the term “about” refers to that variation in the measured quantity as would be expected by the skilled artisan making the measurement and exercising a level of care commensurate with the objective of the measurement and the precision of the measuring equipment used.

As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise. The terms “and/or” and “any combination thereof” and their grammatical equivalents as used herein, can be used interchangeably. These terms can convey that any combination is specifically contemplated. Solely for illustrative purposes, the following phrases “A, B, and/or C” or “A, B, C, or any combination thereof” can mean “A individually; B individually; C individually; A and B; B and C; A and C; and A, B, and C.” The term “or” can be used conjunctively or disjunctively unless the context specifically refers to a disjunctive use.

The recitations of numerical ranges by endpoints include all numbers subsumed within that range (e.g., 1 to 5 includes 1, 1.5, 2, 2.75, 3, 3.80, 4, 5, etc. or 10 or less includes 10, 9.4, 7.6, 5, 4.3, 2.9, 1.62, 0.3, etc.). Where a range of values is “up to”, “at most”, or “at least” a particular value, that value is included within the range.

As used here, “have,” “having,” “include,” “including,” “comprise,” “comprising,” or the like are used in their open-ended sense, and generally mean “including, but not limited to.” It will be understood that “consisting essentially of,” “consisting of,” and the like are subsumed in “comprising” and the like. As used herein, “consisting essentially of,” as it relates to a composition, product, method, or the like, means that the components of the composition, product, method, or the like are limited to the enumerated components and any other components that do not materially affect the basic and novel characteristic(s) of the composition, product, method, or the like.

As used in this specification and claim(s), the words “comprising” (and any form of comprising, such as “comprise” and “comprises”), “having” (and any form of having, such as “have” and “has”), or “including” (and any form of including, such as “includes” and “include”) are inclusive or open-ended and do not exclude additional, unrecited elements or method steps. It is contemplated that any embodiment discussed in this specification can be implemented with respect to any method or composition of the present disclosure, and vice versa. Furthermore, compositions of the present disclosure can be used to achieve methods of the present disclosure. Such inclusive or open-ended words encompass more restrictive or closed terms or phrases, such as “consisting of” or “consisting essentially of.”

Reference in the specification to “some embodiments,” “an embodiment,” “one embodiment,” “embodiments,” “one or more embodiments,” or “other embodiments” means that a particular feature, structure, or characteristic described in connection with the embodiments is included in at least some embodiments, but not necessarily all embodiments, of the present disclosures.

In several places throughout the application, guidance is provided through examples, which examples, including the particular aspects thereof, can be used in various combinations and be the subject of claims. In each instance, the recited list serves only as a representative group and should not be interpreted as an exclusive list. It is to be understood that the particular examples, materials, amounts, and procedures are to be interpreted broadly in accordance with the scope and spirit of the invention as set forth herein.

Reference will now be made in greater detail to various embodiments of the subject matter of the present disclosure, one or more embodiments of which are illustrated in the accompanying drawings. Like numbers used in the figures refer to like components and steps. However, it will be understood that the use of a number to refer to a component in a given figure is not intended to limit the component in another figure labeled with the same number. In addition, the use of different numbers to refer to components in different figures is not intended to indicate that the different numbered components cannot be the same as or similar to other numbered components.

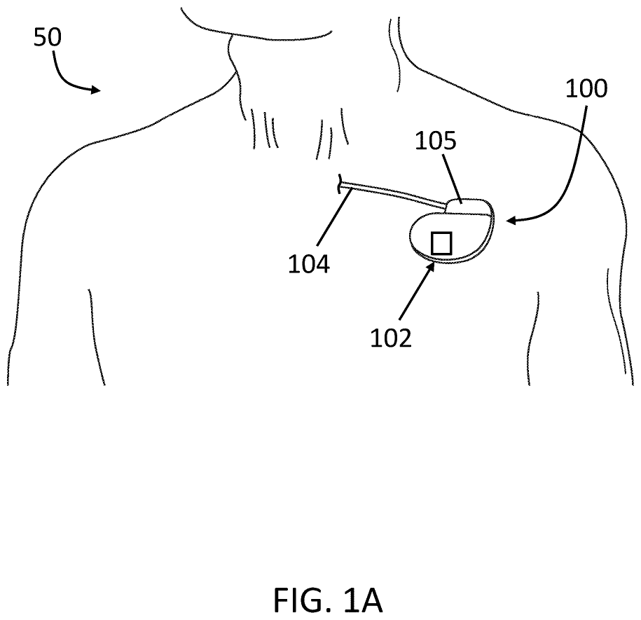

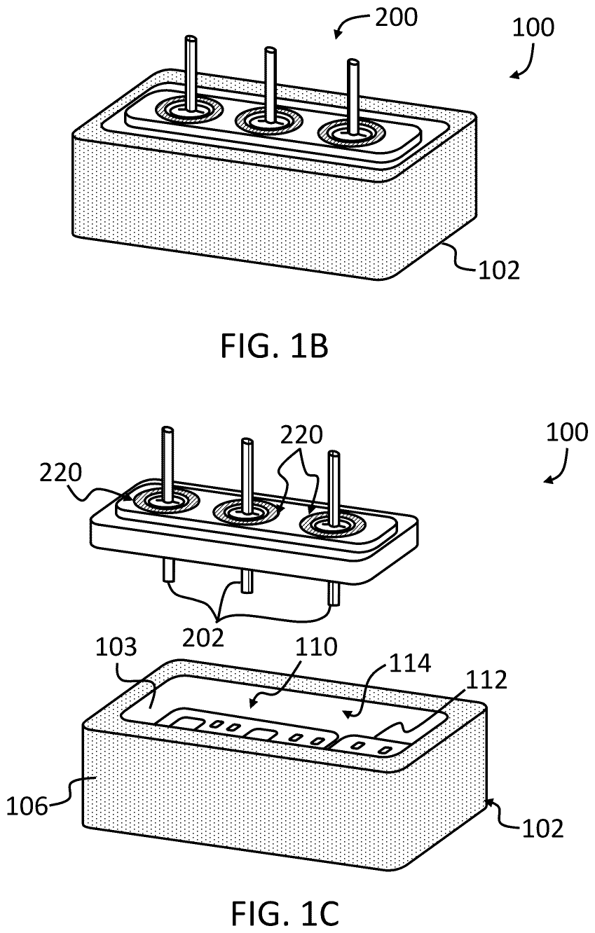

As described herein, substrates and feedthrough assemblies with fluid control beds may be provided, for example, for use in implantable medical devices. An illustrative implantable medical device 100 implanted in a patient 50 including a feedthrough assembly with a fluid control bed is shown in FIG. 1A. The implantable medical device 100 may include a housing 102, which may also be referred to as a case. The housing 102 may define an internal volume of the implantable medical device 100 and may house one or more internal components of the implantable medical device 100, such as one or more batteries, electrical circuits, and the like. The feedthrough assembly may be fixed relative to the housing 102.

The implantable medical device may be configured to be electrically connectable with one or more external components, such as a medical lead 104 (e.g., a lead for delivering therapy or sensing physiological metrics). The one or more external components may be electrically connected to the one or more internal components via a feedthrough assembly, which may be positioned in a connector header 105. The connector header 105 may form a unitary part with, or may be coupled to, the housing 102. The connector header 105 may be configured to receive the one or more external components, such as the lead 104. For example, the connector header 105 may define a bore through which a portion of the lead 104 may be inserted. The lead 104 may have electrical contacts that electrically couple with respective electrical contacts in the connector header 105 when the lead 104 is inserted into the bore. The electrical contacts in the connector header 105 may be electrically coupled to the electrical components of the implantable medical device 100 through one or more feedthrough pins.

Various schematic views of an illustrative embodiment of the implantable medical device 100 (without the connection header 105) that may utilize one or more feedthrough assemblies with a fluid control bed as described herein are shown in FIGS. 1B and 1C. FIG. 1B is a schematic perspective view of the implantable medical device 100 and FIG. 1C is a schematic exploded view of the implantable medical device 100. The implantable medical device 100 may include the housing 102, which may include an inner surface 103 and an outer surface 106. The implantable medical device 100 may also include one or more internal components disposed in the housing 102, such as at least one electronic device 110 and/or a power source 112. In one or more embodiments, the power source 112 can be disposed within a cavity 114 of the housing 102. The power source 112 may include one or more power source contacts that can be operatively coupled to the electronic device 110 or any of one or more feedthrough pins 202.

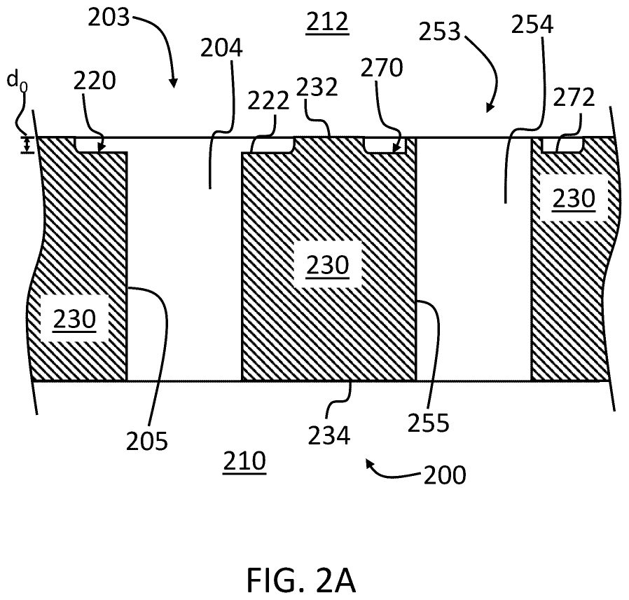

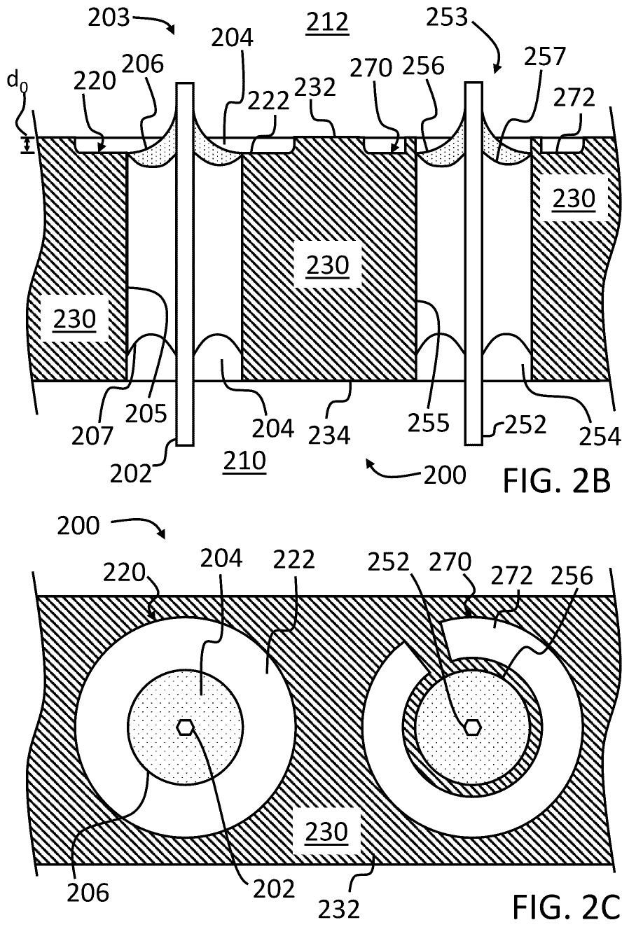

An illustrative feedthrough assembly 200 including a fluid control bed is shown in FIGS. 2A-2C. FIG. 2A shows a cross-sectional side view with certain features omitted for clarity. FIG. 2B shows a cross-sectional side view. FIG. 2C shows a schematic top view. The feedthrough assembly 200 may include a substrate 230, such as a ferrule, having a first major surface 232. The substrate 230 may further have an opposing second major surface 234 (that is, the opposing second major surface 234 may oppose the first major surface 232). The substrate 230 may form a part of an implantable medical device (e.g., the implantable medical device 100), such as a part of the housing (e.g., the housing 102), a ferrule that is bonded to an implantable medical device housing, or the like. The first major surface 232 may be configured to be positioned away from the internal volume 210 of the implantable medical device, which may be toward an external environment 212 (such as within the connector header 105 shown in FIG. 1A, which may define a bore in communication with a patient's bodily fluid). The second major surface 234 may be configured to be positioned towards an internal volume 210 (such as towards the cavity 114 within the housing 102 shown in FIG. 1C) of the implantable medical device.

The feedthrough assembly 200 may include one or more feedthrough cavities, such as the illustrative first feedthrough cavity 203 and second feedthrough cavity 253 shown in FIGS. 2A-2C. Each feedthrough cavity extends into and through the substrate 230 from the first major surface 232 to the second major surface 234 toward the internal volume 210 of the implantable medical device. In at least one embodiment, the substrate 230 defines a feedthrough cavity extending through the substrate 230 from the first major surface 232 to the second major surface 234. The feedthrough cavities 203, 253 each include a feedthrough bore configured to receive a feedthrough pin (e.g., one the feedthrough pins 202). For example, as shown in FIG. 2B, the first feedthrough cavity 203 includes a first feedthrough bore 204 that is defined, at least in part, by a first bore sidewall 205. Likewise, the second feedthrough cavity 253 includes a second feedthrough bore 254 that is defined, at least in part, by a second bore sidewall 255. At least a portion of each feedthrough bore may be disposed between the internal volume 210 and the external environment 212. In embodiments according to the example shown in FIGS. 2B and 2C, the first and second feedthrough bores 204, 254 extend directly from the second major surface 234 toward the first major surface 232. However, in other embodiments, the first and second feedthrough bores 204, 254 extend indirectly from one or both of the first and second major surfaces 232, 234 such that additional features may be included along the feedthrough cavities 203, 253 perimeter at one or both of the first and second major surfaces 232, 234 (e.g., bevels, tappers, countersinks, etc.—not shown). Each feedthrough bore may be tubular or may be of any other suitable shape and contribute to the respective feedthrough cavities.

In one or more embodiments, the substrate 230 includes a fluid control bed on the first major surface 232, which at least partially defines part of a respective feedthrough cavity (e.g., the first feedthrough cavity 203 or the second feedthrough cavity 253), or as discussed further below, at least partially surrounds a portion of the respective feedthrough cavity. In some embodiments, a portion of the substrate 230, such as a portion of the first major surface 232, may completely separate each fluid control bed from the respective feedthrough bore. For example, as shown in FIGS. 2A-2C, the second fluid control bed 270 may be separated from the second feedthrough bore 254 by a portion of the first major surface 232.

In embodiments where the fluid control bed at least partially defines part of a respective feedthrough cavity, the fluid control bed may define one or more channels (e.g., recesses in the first major surface 232) that contribute to the respective feedthrough cavity and may lie in fluid communication with a respective feedthrough bore such that potting material applied within or to the respective cavity/bore may preferentially flow into the one or more channels (e.g., as compared to flowing freely toward or to an adjacent feedthrough cavity. For example, the first fluid control bed 220 defines a first channel 222 that may be in fluid communication with the first feedthrough bore 204 such that both the first channel 222 and the first feedthrough bore 204 collectively form the first feedthrough cavity 203. Similarly, a second fluid control bed 270 defines a second channel 272 within the first major surface 232 such that both the second channel 272 and the second feedthrough bore 254 collectively form the second feedthrough cavity 253. Each fluid control bed may abut the respective feedthrough sidewall such that, for example, the fluid control bed at least partially surrounds the feedthrough sidewall and forms part of a perimeter to the feedthrough bore. Each fluid control bed may be described as defining an area about (e.g., at least partially surrounding) the respective feedthrough bore to contain potting material when such material is deposited, or applied, to or within the feedthrough cavity during assembly of the feedthrough. For example, the first fluid control bed 220 may define an area about the first feedthrough bore 204 to preferentially contain to the first fluid control bed 220, or within the first fluid control bed 220, potting material applied to, or deposited within, the first feedthrough cavity 203. Similarly, as another example, the second fluid control bed 270 may define an area about the second feedthrough bore 254 to contain to the second fluid control bed 270, or within the second fluid control bed 270, potting material applied to, or deposited within, the second feedthrough bore 254.

In at least one embodiment, each fluid control bed defines one or more channels, which may be in fluid communication with a respective feedthrough bore and may be separated from at least one neighboring feedthrough bore by a respective neighboring fluid control bed that likewise defines one or more channels in fluid communication with the neighboring feedthrough bore. In other words, neighboring feedthrough bores may be separated from one another by their respective fluid control beds. For example, as shown in FIGS. 2B and 2C, the first fluid control bed 220 of the first feedthrough bore 204 may be separated from the second feedthrough bore 254 by the second fluid control bed 270 of the second feedthrough bore 254. Likewise, the second fluid control bed 270 of the second feedthrough bore 254 may be separated from the first feedthrough bore 204 by the first fluid control bed 220 of the first feedthrough bore 204.

In some embodiments, a portion of the substrate 230, such as a portion of the first major surface 232, completely separates each fluid control bed from a neighboring fluid control bed. For example, the first fluid control bed 220 may be separated from the second fluid control bed 270 by a portion of the first major surface 232.

In one or more embodiments, the feedthrough assembly 200 further includes one or more feedthrough pins, such as a first feedthrough pin 202 and a second feedthrough pin 252. Each feedthrough pin may be disposed within and extend through the respective feedthrough cavity and bore. For example, the first feedthrough pin 202 may be disposed within and extend through the lumen defined by the first bore sidewall 205 of the first feedthrough bore 204. In some embodiments, each feedthrough pin (e.g., the first feedthrough pin 202) may be coaxially disposed within and extend through the respective feedthrough bore. Each feedthrough pin may be configured to electrically connect a component in the internal volume 210 of the implantable medical device, (such as batteries, capacitors, and/or processors, for example) with a component (such as implantable leads, for example) in communication with an environment external to the implantable medical device.

In some embodiments, the feedthrough assembly 200 includes a potting material 206 disposed between each feedthrough pin and the respective feedthrough bore, or the respective feedthrough bore sidewall. The potting material 206 may aid in electrically insulating the feedthrough pin, for example, from the substrate. While the potting material 206 generally adheres to surfaces of the feedthrough assembly (e.g., surfaces of the substrate, the insulating plugs, the feedthrough pins, the bore sidewall, etc.), delamination of the potting material from such surfaces may reduce the insulative effect of the potting material. Furthermore, in cases where potting material of one feedthrough bore/cavity interconnects with potting material of an adjacent feedthrough bore/cavity, delamination may spread between the feedthrough bores/cavities. As illustrated in FIGS. 2B and 2C, a first potting material 206 may be disposed between the first feedthrough pin 202 the first feedthrough bore sidewall 205 such that the potting material 206 is at least partially within the first feedthrough cavity 203. The potting material 206 may be positioned to help separate elements of the feedthrough assembly 200 from the external environment 212 and/or to supplement electrical insulation of the first and second feedthrough pins 202, 252. For example, the first potting material 206 may be positioned to help separate the internal volume 210, portions of the first feedthrough pin 202, and/or portions of the first feedthrough bore 204 from the external environment 212.

The feedthrough assembly 200 may include an insulating plug disposed at least partially within each feedthrough bore, such as the insulating plugs 207, 257 disposed respectively within the first and second feedthrough bores 204, 254 in FIGS. 2A and 2B. The insulating plugs 207, 257 may be configured and positioned to provide a hermetic seal between the external environment 212 and the internal volume 210. The insulating plugs 207, 257 may be positioned to separate the potting material 206 from the internal volume 210. The insulating plugs 207, 257 may be brazed to provide a hermetic seal. Each of the feedthrough pins 202, 252 may extend through a respective lumen defined by the respective insulating plug 207, 257.

It will be understood in view of this disclosure that sealing and insulating capability (e.g., hermetic sealing capability, electrical insulating capability, etc.) of the feedthrough assembly 200 may be provided by one or more of a combination of insulating elements, including the insulating plugs, the brazing, and the potting material described herein. Further insulating elements may contribute to (e.g., provide) the sealing and insulating capabilities. Further insulating elements may include, for example, an insulating material applied (e.g., adhered) to one or more portions of the feedthrough assembly (e.g., to the first major surface 232 of the substrate 230, to the pins 202, 252, to the potting material 206, 256, etc.). Such additional insulating elements may include any suitable insulating material. Suitable insulating materials may include, as just one example, liquid silicone rubber. In some embodiments, the insulating material may not sufficiently adhere directly to one or more portions of the feedthrough assembly. In such embodiments an adhesive capable of adhering to both the substrate and the insulating material may be used. In other words, the adhesive may be used to bond the insulating material to the substrate. For example, in an illustrative embodiment including a grade 5 titanium substrate and a liquid silicone rubber insulating material (which is not capable of sufficiently adhere directly to grade 5 titanium), a medical adhesive may be used to bond the liquid silicone rubber to the substrate.

The potting material (e.g., potting materials 206, 256) may be or include any suitable materials or combination of materials. Suitable potting materials may be selected based on biocompatibility, electrical insulation, viscosity, curing characteristics (e.g., curing time, photocurability, thermocurability, etc.), adhesion to and/or material compatibility with the feedthrough sidewall, adhesion to and/or material compatibility with the feedthrough pin, and adhesion to and/or material compatibility with the substrate, as examples. As another example, suitable potting materials may be selected on desired curing temperature. Suitable potting materials may include polymeric materials such as epoxies, adhesives, thermoplastics, or rubber, as examples. It will be understood in light of the present disclosure that any suitable potting materials or combination of potting materials may be used, and the disclosure is not limited in this regard. It will be further understood in light of the present disclosure that suitable potting materials may vary depending on factors, including those described herein.

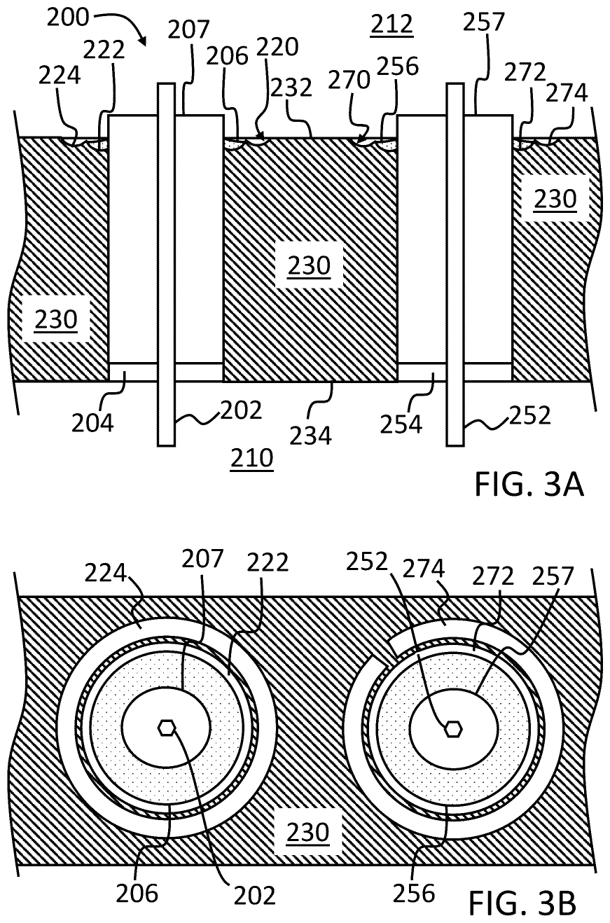

The insulating plug (e.g., insulating plugs 207, 257) may be, or include, any suitable materials or combination of materials and may be, or include any suitable configuration. Suitable insulating plugs may include glass, metal, metal alloy, or ceramic, as examples. The insulating plugs may each be brazed (e.g., sealed with molten metal, such as gold) or glassed (e.g., sealed with molten class) to provide a hermetic seal. Furthermore, suitable insulating plugs may be, or include, recessed insulating plugs (as shown, for example, in FIG. 2B) or protruding insulating plugs (as shown, for example, in FIG. 3A). It will be understood in light of the present disclosure that any suitable insulating plugs, including any suitable insulating plug materials or combination of materials, may be used, and the disclosure is not limited in this regard. It will be further understood in light of the present disclosure that suitable insulating plugs may vary depending on factors, including those described herein.

In one or more embodiments, the fluid control bed defines at least one fluid control channel, which may be in fluid communication with and at least partially surround the respective feedthrough bore. For example, and as illustrated in FIGS. 2B and 2C, the first fluid control bed 220 of the first feedthrough bore 204 may define a fluid control channel 222 abutting and fully surrounding the first feedthrough bore 204. Similarly, and as illustrated in FIGS. 2B and 2C, the second fluid control bed 270 of the second feedthrough bore 254 may define a fluid control channel 272 abutting and partially surrounding the second feedthrough bore 254.

In some embodiments, the fluid control bed defines a plurality of channels including an inner channel (e.g., the channels 222, 272) and an outer channel, which may be in fluid communication with and at least partially surround the inner fluid control channel. For example, the illustrative feedthrough assembly 200 with the first fluid control bed 220 further defining an outer channel 224 abutting and fully surrounding the fluid control channel 222 is shown in FIGS. 3A and 3B. As another example, and as also shown in FIGS. 3A and 3B, the second fluid control bed 270 may define an outer channel 274 abutting and partially surrounding the fluid control channel 272.

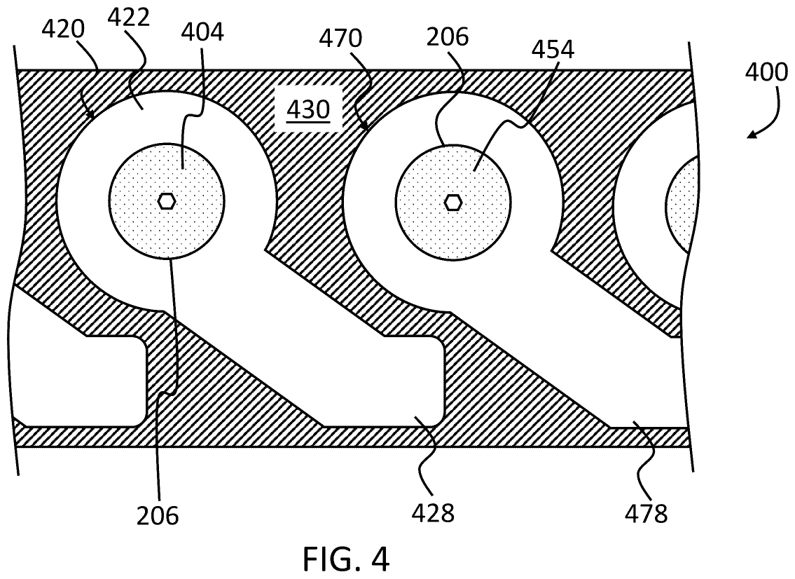

In some embodiments, the fluid control bed defines an auxiliary channel. A top view of an illustrative feedthrough assembly 400 including auxiliary channels is shown in FIG. 4. The illustrative feedthrough assembly 400 includes a first fluid control bed 420 on a major surface 430 (e.g., similar to the first major surface 232) and a second fluid control bed 470 on the major surface 430. The first fluid control bed 420 may define a fluid control channel 422 in fluid communication with and at least partially surrounding a first feedthrough bore 404. The first fluid control bed 420 may further define an auxiliary channel 428 in fluid communication with the fluid control channel 422 and configured to control the flow and/or wetting of the potting material 206 (e.g., potting adhesive) from or for the first feedthrough bore 404. For example, the auxiliary channel 428 may abut and connect with a portion of the fluid control channel 422. The second fluid control bed 470 may similarly include a second auxiliary channel 478.

Additionally or alternatively to having one or more channels (e.g., the fluid control channels 222, 272, 422, 472 the outer channels 224, 274, and/or the auxiliary channels 428, 478), in some embodiments, the fluid control bed at least partially defining a respective feedthrough cavity includes a surface texture, such as a bed fluid control surface texture on the first major surface 232. The surface textures (e.g., the bed fluid control surface textures) may be configured to modify interactions between the fluid control bed and a fluid, as compared with interactions between the fluid and other portions of the substrate. For example, the surface textures (e.g., the bed fluid control surface textures) may be configured to improve the wettability of the fluid control bed. More specifically, the fluid control surface textures may be configured to control the wetting and flow of the potting material (e.g., a potting adhesive). Control of the wetting and flow of the potting material may be desirable to prevent fluid (e.g., the potting material) of one feedthrough bore (e.g., the potting material 206 provided in the first feedthrough bore 204) from interconnecting with fluid (e.g., the potting material) of a neighboring feedthrough bore (e.g., the potting material 256 provided in the second feedthrough bore 254), or from interconnecting with the neighboring feedthrough bore, itself. Surface textures, such as the bed fluid control surface textures may be described as textured (e.g., grooved or roughened) compared to adjacent portions of the first major surface such that the potting material preferentially flows over the textured surface of the textured fluid control bed, as opposed to the comparatively smooth surface of the adjacent portions of the first major surface.

In at least one embodiment, the feedthrough pin includes a pin fluid control exterior surface texture. Similarly to the bed fluid control surface texture, the pin fluid control exterior surface texture may be configured to affect the wettability of the feedthrough pin. More specifically, the pin fluid control exterior surface texture may be configured to control the wetting and flow of the potting material (e.g., a potting adhesive). The pin fluid control surface textures may be described as textured (e.g., grooved or roughened) relative to adjacent, comparatively smoother, portions of the pin surface.

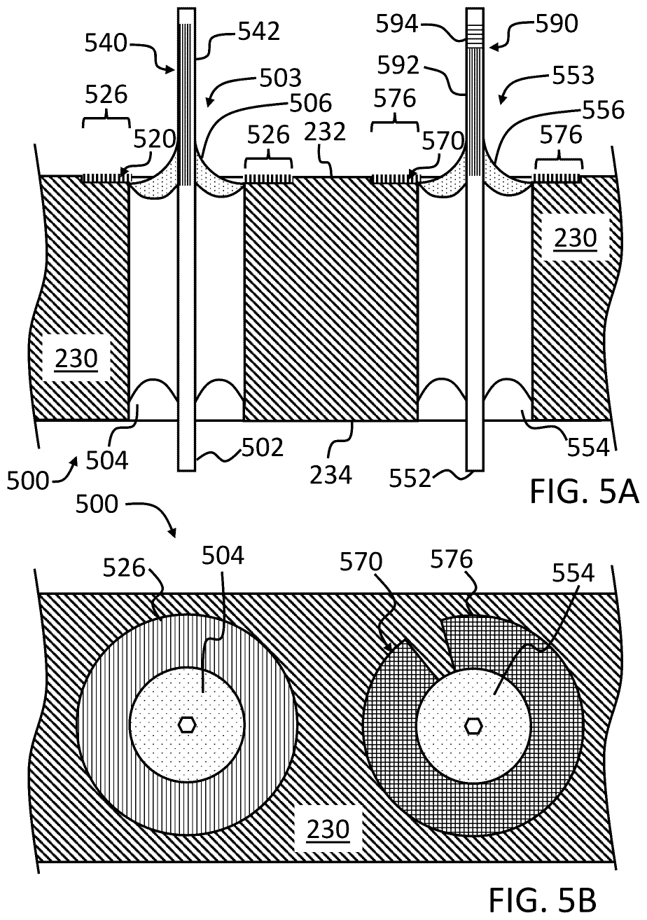

An illustrative feedthrough assembly 500 including fluid control beds having bed fluid control surface textures and including feedthrough pins having pin adhesive control exterior surface textures is shown in FIGS. 5A and 5B. The feedthrough assembly 500 may further include potting adhesive 506, 556, as described herein. As an example of a bed fluid control surface texture, a first fluid control bed 520 at least partially defines a first feedthrough cavity 503 and at least partially surrounds a portion of the first feedthrough cavity 503. The first fluid control bed 520 may include a first bed fluid control surface texture 526. In one or more embodiments, the first bed fluid control surface texture 526 may include a plurality of grooves. As an example, the first bed fluid control surface texture 526 is shown in FIGS. 5A and 5B with a plurality of grooves that are substantially parallel (e.g., parallel or nearly parallel) to each other. Without wishing to be bound by theory, substantially parallel grooves may be desirable to control wetting and flow of potting material. In particular, substantially parallel grooves may be desirable to wick potting material (such as overflowed potting adhesive) along the grooves, or in a direction parallel to the grooves. Substantially parallel grooves may additionally or alternatively be desirable to prevent formation of a drop of potting material that may subsequently fall, travel, or otherwise undergo uncontrolled movement on a substrate (e.g., the substrate 230) and/or to neighboring feedthrough bores. The bed fluid control surface textures may be used in addition to, or in place of, the one or more defined channels discussed above with respect to FIGS. 2A-3B.

As another example of a bed fluid control surface texture, and as shown in FIGS. 5A and 5B, a second fluid control bed 570 at least partially defines a second feedthrough cavity 553. The second fluid control bed 570 includes a second bed fluid control surface texture 576 defining a first plurality of substantially parallel (e.g., parallel or nearly parallel) grooves and a second plurality of substantially parallel grooves that are substantially perpendicular (e.g., perpendicular or nearly perpendicular) to the first plurality of substantially parallel grooves. In other words, the first plurality of substantially parallel grooves and the second plurality of substantially parallel grooves may form a cross-hatched pattern of intersecting grooves. Such a pattern of intersecting pluralities of parallel grooves may be desirable to contain flow of potting material (such as overflowed potting adhesive) to a defined region, or area. For example, the second bed fluid control texture 576 including the first plurality of substantially parallel grooves and the second plurality of substantially parallel grooves that are substantially perpendicular to the first plurality of substantially parallel grooves may be desirable to contain flow of potting material to the second fluid control bed 570 and, thus, away from the first feedthrough bore 504 and the first fluid control bed 520.

As an example of a pin fluid control exterior surface texture, and as shown in FIG. 5A, a first feedthrough pin 502 may include a first pin fluid control exterior surface texture 540. The first pin fluid control exterior surface texture 540 may include a plurality of axial grooves 542 to draw the potting material from the first feedthrough bore 504. Drawing the potting material from the first feedthrough bore 504 may be desirable, for example, to control or prevent the flow of liquid potting material from the first feedthrough bore 504 by directing the flow up the shaft of the pin. The plurality of axial grooves 542 may be disposed radially about the pin first feedthrough 502, (e.g., distributed radially about the entire circumference of the pin or distributed radially about a partial circumference of the pin). The plurality of axial grooves 542 may extend axially along the first feedthrough pin 502, for example, from the first feedthrough bore 5404 away from the first feedthrough bore 504.

In some embodiments, the pin fluid control exterior surface texture includes a stop, such as at least one radial groove, to stop the flow of the potting material along the pin fluid control exterior surface texture. For example, the second feedthrough pin 552 may include a second pin fluid control exterior surface texture 590 including a plurality of axial grooves 592 to draw the potting material from the second feedthrough bore 554 and a plurality of radial grooves 594 to serve as a stop. The stop may be desirable, for example, to stop the flow of the potting material along the plurality of axial grooves 592 and up the shaft of the second feedthrough pin 552.

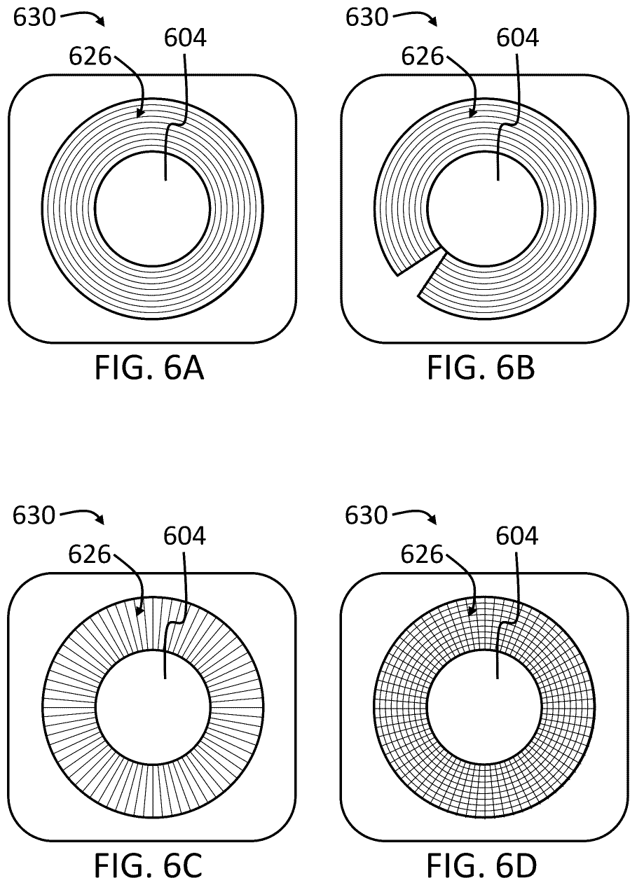

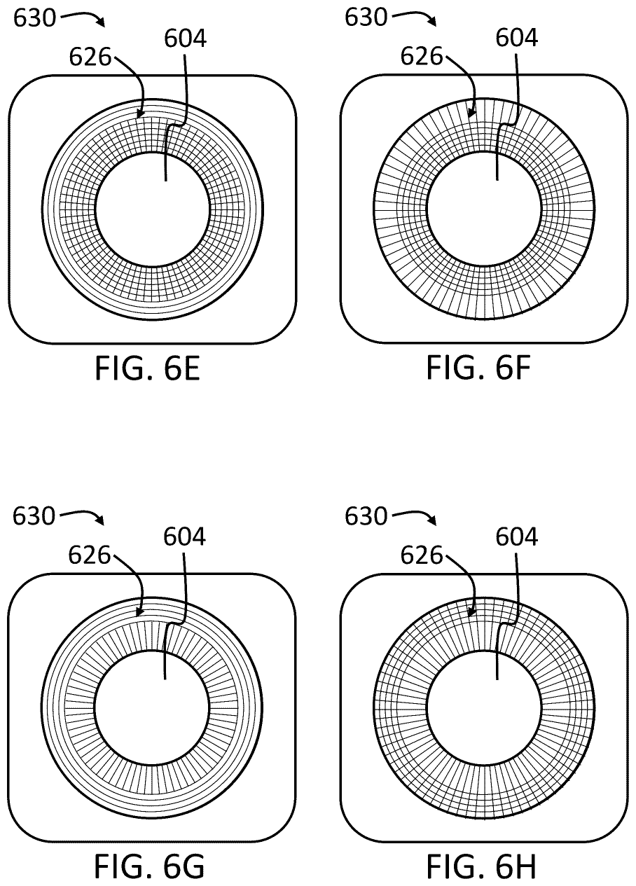

Illustrative substrates for forming a feedthrough assembly (e.g., the feedthrough assemblies 200, 500) for an implantable medical device (e.g., the implantable medical device 100) including additional or alternative illustrative bed fluid control surface textures is shown in FIGS. 6A-6H. Illustrative bed fluid control surface textures may include a plurality of substantially concentric (e.g., concentric or nearly concentric) grooves at least partially surrounding the respective feedthrough bore. For example, the illustrative substrate 630 including a bed fluid control surface texture 626 abutting a feedthrough bore 604, and the bed fluid control surface texture 626 may have a plurality of substantially concentric grooves surrounding the feedthrough bore 604, as shown in FIG. 6A. As another example, the illustrative substrate 630 may have a bed fluid control surface texture 626 with a plurality of substantially concentric grooves partially surrounding the feedthrough bore 604, as shown in FIG. 6B.

In some embodiments, illustrative bed fluid control surface textures include a plurality of axial grooves substantially normal (e.g., normal or nearly normal) to the feedthrough bore and at least partially surrounding the feedthrough bore. For example, the illustrative substrate 630 may include the bed fluid control surface texture 626 having a plurality of axial grooves substantially normal to the feedthrough bore 604 and surrounding the feedthrough bore 604, as shown in FIG. 6C. As still another example, the illustrative substrate 630 may include the bed fluid control surface texture 626 having a first plurality of axial grooves substantially normal to the feedthrough bore 604 and further having a second plurality of substantially concentric grooves intersecting with the first plurality of axial grooves, as shown in FIG. 6D.

In further examples, the illustrative substrate 630 includes the bed fluid control surface texture 626 having a combination of axial grooves and concentric grooves, as shown in FIGS. 6E-6F. As shown in FIG. 6E, the bed fluid control surface texture 626 may have an outer plurality of concentric grooves at least partially surrounding an inner plurality of intersecting axial grooves and concentric grooves. As shown in FIG. 6F, the bed fluid control surface texture 626 may have an outer plurality of axial grooves at least partially surrounding an inner plurality of intersecting axial grooves and concentric grooves. As shown in FIG. 6G, the bed fluid control surface texture 626 may have an outer plurality of concentric grooves at least partially surrounding an inner plurality of axial grooves. As shown in FIG. 6H, the bed fluid control surface texture 626 may have an outer plurality of intersecting axial grooves and concentric grooves at least partially surrounding a plurality of inner axial grooves.

It will be understood in light of the present disclosure that any number and combination of bed fluid control surface textures and/or pin fluid control exterior surface textures may be useful, additionally or alternatively to the illustrative textures shown in the figures. For example, in some embodiments, the bed fluid control surface texture may be, or include a groove in the first major surface (e.g., the first major surface 232) of the substrate that forms a spiral about the feedthrough bore (e.g., the first feedthrough bore 204). As another example, the bed fluid control surface texture may be, or include, a groove in the major surface of the substrate that forms a spiral about the feedthrough bore and forms a plurality of grooves in the major surface that axially extend substantially normal from the feedthrough bore. As still another example, the pin fluid control exterior surface texture may be, or include, a groove that forms a helix about the circumference and along the shaft of the feedthrough pin. In still yet another example, the pin fluid control exterior surface texture may be, or include, a plurality of grooves that forms a plurality of helices extending along the shaft and about the circumference of the feedthrough pin.

In one or more embodiments, as described herein, each of the bed fluid control surface texture and the pin fluid control exterior surface texture includes a surface roughness. Each of the bed fluid control surface texture and the pin fluid control exterior surface texture may have any suitable roughness value. Roughness of the surface may be quantified, for example, as Ra (profile roughness), Sa (area roughness), grit, or root mean square (RMS). Roughness may be measured, for example, using confocal laser scanning microscopy (CLSM). Suitable roughness values may be selected based on factors such as desired wettability and/or flow control of the potting material, material compatibility with the substrate, material compatibility with the pin, desired surface area of the texture/bed, or relative thickness/depth of the fluid control bed, as a few examples. Suitable Sa (area roughness) values may include, for example, between 2 micrometers (um) and 10 um or between 3 um and 8 um. In one embodiment, the Sa of the pin fluid control surface texture or the bed fluid control surface texture may be approximately 5 um. It will be understood in light of the present disclosure that any suitable roughness may be used, and the disclosure is not limited in this regard. Furthermore, suitable roughness may be selected based on factors, such as those discussed herein.

In one or more embodiments, as described herein, each of the bed fluid control surface texture and the pin fluid control exterior surface texture may include a plurality of grooves. Each (i.e., any one or more) groove may independently have any suitable depth. Suitable groove depths may be selected based on factors such as desired wettability and/or flow control of the potting material, material compatibility with the substrate, material compatibility with the feedthrough pin, radius of the grooves (e.g., for circular or spiral grooves), desired surface area of the texture/bed, technique used to form the texture, desired sharpness of the groove peaks, or relative thickness/depth of the fluid control bed, as a few examples. Suitable groove depths may include, for example, between 10 um and 20 um or between 5 um and 40 um. In one embodiment, the groove depths may be approximately 15 um. As further examples, suitable groove depths may include substantially equal (e.g., equal or nearly equal) to the respective fluid control bed depth, the respective fluid control bed depth or greater, and the respective fluid control bed depth or less. It will be understood in light of the present disclosure that any suitable groove depth may be used, and the disclosure is not limited in this regard. Furthermore, suitable groove depths may be selected based on factors, such as those discussed herein.

The plurality of grooves may have any suitable minimum-to-minimum period. The minimum-to-minimum period may be described as an average of the distance between groove minimums. In particular, the minimum-to-minimum period may be described as an average of the distance from the deepest part of each groove to the deepest part of a respective adjacent groove, where each distance is measured along an axis perpendicular to the grooves. Suitable groove periods may be selected based on factors such as desired wettability and/or flow control of the potting material, material compatibility with the substrate, material compatibility with the feedthrough pin, technique used to form the texture, desired sharpness of the groove peaks, or relative thickness/depth of the fluid control bed, as a few examples. As another example, suitable groove periods may be selected based on material properties of the potting material, such as viscosity, flow rate, or pot life. Suitable minimum-to-minimum periods may include, for example, between 10 um and 120 um or between 20 um and 100 um. In one embodiment, the minimum-to-minimum period may be approximately 20 um. It will be understood in light of the present disclosure that any suitable groove period may be used, and the disclosure is not limited in this regard. Furthermore, suitable groove periods may be selected based on factors, such as those discussed herein.

While suitable minimum-to-minimum periods and factors for selecting minimum-to-minimum periods are discussed herein, it will be understood in light of the present disclosure that any suitable maximum-to-maximum periods may similarly be used and, further, that maximum-to-maximum periods may be selected based on similar factors (e.g., the same factors or nearly the same factors) as described herein with respect to minimum-to-minimum periods. Similarly, any suitable spacing between grooves may be used. The spacing between grooves may be described as a distance between the minimum (or maximum) of one groove and the respective minimum (or maximum) of an adjacent groove, where the distance is measured along an axis perpendicular to the grooves. Spacing between grooves may be selected based on similar factors as described herein with respect to minimum-to-minimum periods.

The plurality of grooves may have any suitable groove aspect ratios. Groove aspect ratios may be described as a ratio between groove depth and groove period, or as a ratio between groove depth and groove spacing. Suitable groove aspect ratios may be selected based on factors such as such as desired wettability and/or flow control of the potting material, material compatibility with the substrate, material compatibility with the feedthrough pin, technique used to form the texture, desired sharpness of the groove peaks, or relative thickness/depth of the fluid control bed, as a few examples. As another example, suitable groove aspect ratios may be selected based on material properties of the potting material, such as viscosity, flow rate, or pot life. Suitable groove aspect ratios may include, for example, between 1:1 and 1:5 or between 1:2 and 1:4. In one embodiment, the groove aspect ratio may be approximately 1:2. It will be understood in light of the present disclosure that any suitable groove aspect ratios may be used, and the disclosure is not limited in this regard. Furthermore, suitable groove aspect ratios may be selected based on factors, such as those discussed herein.

In some embodiments, as described herein, the fluid control bed has a depth. The fluid control bed depth (e.g., the depth do in FIG. 2B) may be defined as a maximum dimension of the fluid control bed measured from the major surface (e.g., the major surface 232) toward the internal volume (e.g., the internal volume 210) and perpendicular to the major surface. The fluid control bed may have any suitable depth. Suitable fluid control bed depths may be selected based on factors such as relative depth/period of the plurality of grooves, dimensions (e.g., diameter) of the respective feedthrough bore, dimensions (e.g., diameter) of the feedthrough pin, or dimensions (e.g., diameter) of the lead (e.g., the lead 104), as some examples. As another example, suitable fluid control bed depths may be selected based on material properties of the potting material, such as viscosity, flow rate, curvature of meniscus, or pot life. Suitable fluid control bed depths may be, for example, between 10 um and 20 um or between 5 um and 40 um. In one embodiment, the fluid control bed depth may be approximately 15 um. It will be understood in light of the present disclosure that any suitable fluid control bed depth may be used, and the disclosure is not limited in this regard. Furthermore, suitable fluid control bed depths may be selected based on factors, such as those discussed herein.

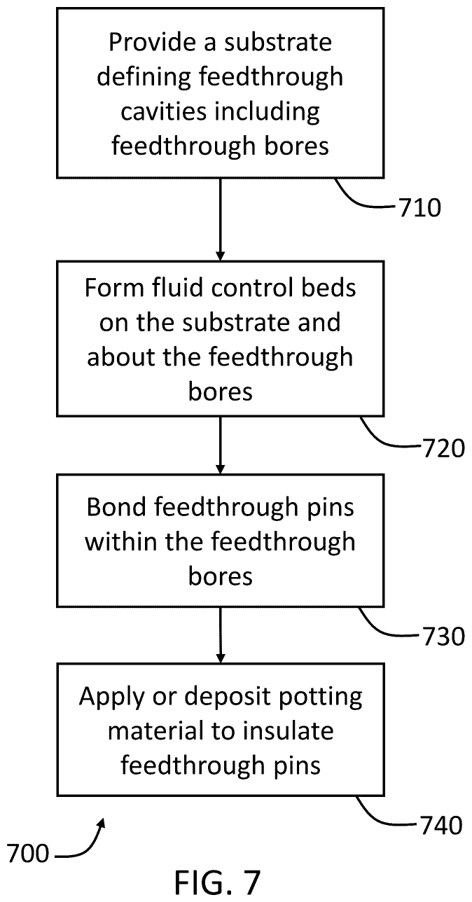

A method for forming an implantable medical device feedthrough assembly including fluid control beds according to illustrative embodiments described herein may be provided. An illustrative method 700 of forming a feedthrough assembly (e.g., the feedthrough assembly 200) is shown in FIG. 7. The method 700 may include providing 710 a substrate (e.g., the substrate 230) having a first major surface (e.g., the major surface 232) and an opposing second major surface (e.g., the second major surface 234), the substrate defining at least first and second feedthrough cavities (e.g., the first and second feedthrough cavities 203, 253) extending through the substrate from the first major surface to the second major surface and each including a respective feedthrough bore (e.g., the first and second feedthrough bores 204, 254).

In one or more embodiments, the method 700 includes forming 720 at least first and second fluid control beds (e.g., the first and second fluid control beds 220, 270) on the first major surface. The first and second fluid control beds may each form an area about the respective feedthrough bore to contain potting material to, or within, the respective feedthrough cavity. In particular, each fluid control bed may be configured to contain potting material applied to, or deposited within, the respective feedthrough bore. For example, the first fluid control bed may form an area about the first feedthrough bore to preferentially contain within the first feedthrough cavity potting material applied to, or deposited within, the first feedthrough bore. Similarly, the second fluid control bed may form an area about the second feedthrough bore to preferentially contain within the first feedthrough cavity potting material applied to, or deposited within, the first feedthrough bore. Forming 720 each fluid control bed may include forming a fluid control channel (e.g., the first fluid control channel 222) abutting and at least partially surrounding the respective feedthrough bore. Forming 720 the at least first and second fluid control beds may include any suitable technique. Suitable techniques may include, for example, bead blasting, laser etching, laser spot-welding, machining, micro-etching, pulsed laser micro-machining the major surface, femtosecond laser machining the major surface, laser drilling the major surface, laser drilling, laser milling, laser chemical machining, direct laser writing, laser assisted machining, laser-induced breakdown, laser chemical vapor deposition, ultrasonic machining, electrochemical, machining, polishing, buffing, broaching, honing, electron beam machining, wire electrical discharge machining, abrasive flow machining, or a combination of two or more thereof.

Furthermore, forming 720 each fluid control bed may include forming an outer channel (e.g., the outer channel 224) abutting and at least partially surrounding the fluid control channel. In at least one embodiment, the forming 720 at least first and second fluid control beds may include forming a bed fluid control surface texture (e.g., the bed fluid control surface texture 226) comprising a plurality of grooves, as described herein. Forming the fluid control beds may result in surface texturing of the fluid control beds or the fluid control beds may be subjected to further processing to provide the surface texturing.

In some embodiments, the method 700 includes bonding 730 at least first and second feedthrough pins (e.g., the first and second feedthrough pins 202, 252) within the respective first and second feedthrough bores, for example, using brazing or glassing. The bonding 730 the first and second feedthrough pins may form a hermetic seal, as described herein.

In one or more embodiments, the method 700 includes applying 740, or depositing, potting material to or within the first and second feedthrough bores to insulate (e.g., additionally insulate or supplementarily insulate) the first and second feedthrough pins.

While fluid control beds are primarily described herein as each forming an area about a respective feedthrough bore, it will be understood in view of this disclosure that fluid control beds may additionally or alternatively form an area about (e.g., between, at least partially surrounding, etc.) a plurality of feedthrough bores (e.g., one or more, two or more, three or more, or four or more feedthrough or an area about (e.g., between, at least partially surrounding, etc.) a plurality of feedthrough cavities (e.g., one or more, two or more, three or more, or four or more feedthrough cavities).

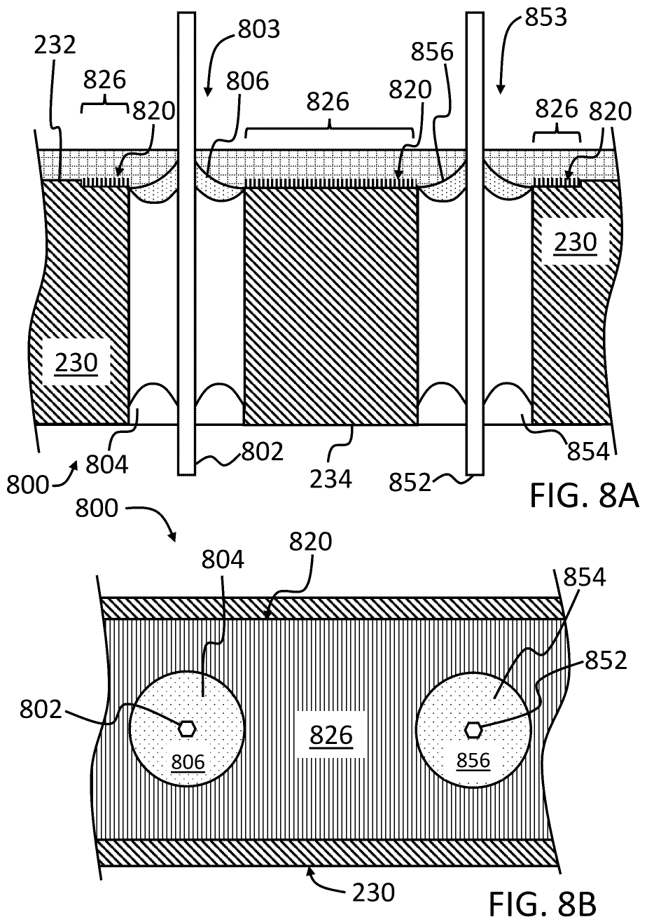

In some embodiments, for example, a fluid control bed may include a bonding surface texture. An illustrative feedthrough assembly 800 including a fluid control bed 820 with a bonding surface texture is shown in FIGS. 8A and 8B. FIG. 8A shows a cross-sectional side view and FIG. 8B shows a schematic top view with certain features omitted for clarity. The feedthrough assembly 800 may include a substrate 230, such as a ferrule, having a first major surface 232 and an opposing second major surface 234. The feedthrough assembly 800 may further include potting adhesive 806, 856, as described herein. The substrate 230 may form a part of an implantable medical device (e.g., the implantable medical device 100), such as a part of the housing (e.g., the housing 102), a ferrule that is bonded to an implantable medical device housing, or the like. The feedthrough assembly 800 may include one or more feedthrough cavities, such as a first feedthrough cavity 803 and a second feedthrough cavity 853. The feedthrough cavities 803, 853 respectively include a first feedthrough bore 804 and a second feedthrough bore 854, each configured to receive, respectively, a first feedthrough pin 802 and a second feedthrough pin 852.

In one or more embodiments, the substrate 230 includes the fluid control bed 820 having a surface texture on the first major surface 232. As described herein, the surface texture may be configured to modify interactions between the fluid control bed 820 and a fluid, as compared with interactions between the fluid and other portions of the substrate 230. For example, as described herein, the surface texture may be a fluid control surface texture (e.g., the first bed fluid control surface texture 526). As another example, and as shown in FIGS. 8A and 8B, the surface texture may be a bonding surface texture 826. A bonding surface texture may be described as a surface texture configured to increase surface area of the fluid control bed capable of interaction with a fluid (e.g., medical adhesives, potting materials, etc.). Bonding surface textures may advantageously increase the extent of interaction between a liquid adhesive and the fluid control bed, such as by increasing the surface area of the fluid control bed capable of interaction (e.g., interface) with the liquid adhesive to form bonds therebetween, increasing tortuosity of a bond interface between the liquid adhesive and the fluid control bed, and/or increasing an extent of mechanical interlock between the liquid adhesive and the fluid control bed. Bonding surface textures providing increased surface area, tortuosity, and/or mechanical interlock of the fluid control bed may advantageously improve bonding efficacy, such as by improving the strength and/or durability of bonds.

For example, as shown in FIG. 8A, the feedthrough assembly 800 may include a medical adhesive 840. The medical adhesive 840 may be used to improve (e.g., promote, provide, etc.) fluid and/or electrical isolation for portions of the feedthrough assembly 800. In some embodiments, medical adhesive is used to improve (e.g., promote, provide, etc.) bonding between the substrate 230 and an insulating material (not shown), such as liquid silicone rubber, as described herein. By improving bonding between the substrate 230 and the medical adhesive 840, bonding of the insulating material to the feedthrough assembly 800 may be improved.

The bonding surface texture 826 may include any suitable feature or combination of features. Suitable bonding surface texture features may include, for example, patterns, grids, grooves, dimples, and the like. Suitable textures may include the fluid control surface textures described herein (e.g., the first bed fluid control surface texture 526, the second bed fluid control surface texture 576, the plurality of axial grooves 542, the plurality of radial grooves 594, the bed fluid control surface textures 626, etc.). It will be understood in view of this disclosure that suitable bonding surface texture features, such as those described herein, may be suitable as fluid control surface texture features. Similarly, it will be understood in view of this disclosure that suitable fluid control surface texture features may be suitable as bonding surface texture features. It will be further understood in view of this disclosure that suitable fluid control surface texture characteristics such as surface roughness, texture depths (e.g., groove depths), minimum-to-minimum periods, aspect ratios (e.g., groove aspect ratios), and the like, as described herein, may be suitable as characteristics of bonding surface textures, and vice versa.

The fluid control bed 820 may define a bed surface area, which may be described as a surface area of the fluid control bed 820 without the bonding surface texture 826, or as a surface area of a plane (e.g., on a Euclidian plane, a hyperbolic plane, an elliptic plane, etc.) defined by the fluid control bed (i.e. an area measured without considering surface textures, or with a relatively high level of generalization, such as using units of 1 square millimeter or greater, 500 square micrometer or greater, or 100 square micrometer or greater). The fluid control bed 820 may define a texture surface area (e.g., a bonding surface area), which may be described as a surface area of the fluid control bed 820 (i.e., an area measured considering surface textures, or with a relatively low level of generalization, such as using units of 1 square micrometer or less, 500 square nanometers or less, or 100 square nanometers or less).

As described herein, surface textures, such as the bonding surface textures 826, may advantageously increase the surface area (e.g., bonding surface area) of the fluid control bed 820. In particular, surface textures may advantageously increase the surface area (e.g., bonding surface area) of the fluid control bed 820 relative to the surface area of a comparable fluid control bed without a surface texture, or relative to a bed surface area. The fluid control bed may have any suitable texture surface area. Suitable texture surface areas may include, for example, 1.5 times or greater, 2 times or greater, 3 times or greater, 4 times or greater, 5 times or greater, 6 times or greater, 7 times or greater, 8 times or greater, 9 times or greater, or 10 times or greater the bed surface area.

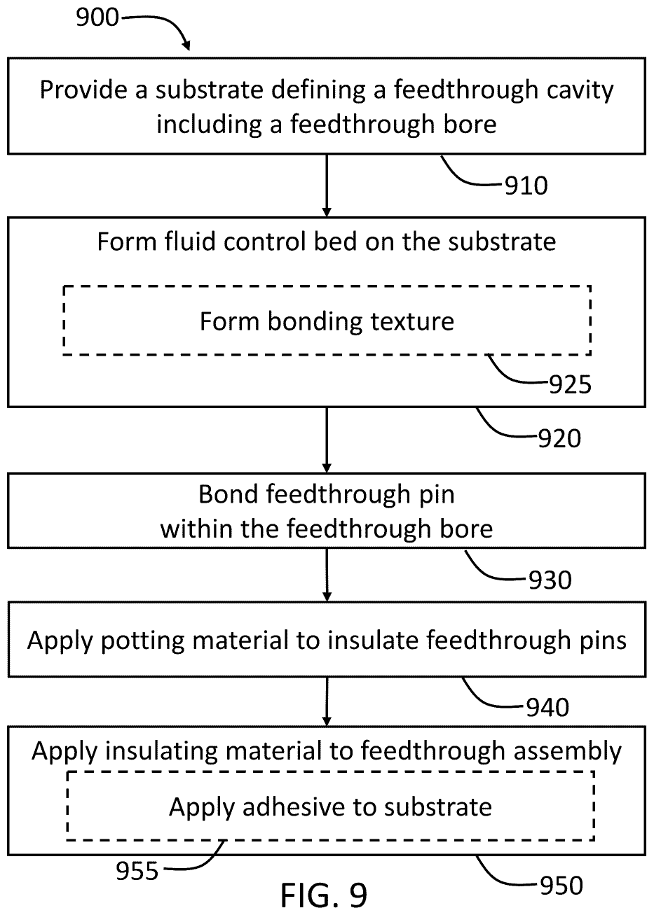

A method for forming an implantable medical device feedthrough assembly including a fluid control bed with a bonding surface texture according to illustrative embodiments described herein may be provided. An illustrative method 900 of forming a feedthrough assembly (e.g., the feedthrough assembly 800) is shown in FIG. 9. The method 900 may include providing 910 a substrate (e.g., the substrate 230) having a first major surface (e.g., the major surface 232) and an opposing second major surface (e.g., the second major surface 234), the substrate defining at least one feedthrough cavity (e.g., the feedthrough cavity 803) extending through the substrate from the first major surface to the second major surface and including a respective feedthrough bore (e.g., the feedthrough bore 804).

In one or more embodiments, the method 900 includes forming 920 a fluid control bed (e.g., the fluid control bed 820) on the first major surface (e.g., the first major surface 232). The fluid control bed may form an area about (e.g., between, at least partially surrounding, etc.) the at least one feedthrough cavity, the area configured to increase surface area of the fluid control bed capable of interaction with a fluid. Forming 920 the fluid control bed may include optionally forming 925 a bonding surface texture (e.g., the bonding surface texture 826).

In some embodiments, the method 900 includes bonding 930 at least one feedthrough pin within the at least one feedthrough bore, for example, using brazing or glassing.

In one or more embodiments, the method 900 includes applying 940 (e.g., depositing) potting material to or within the feedthrough bore to insulate (e.g., additionally insulate or supplementarily insulate) the feedthrough pin.

In at least one embodiment, the method 900 includes applying 950 (e.g., depositing, spreading, injecting, adhering, etc.) an insulating material (e.g., liquid silicone rubber) to one or more portions of the feedthrough assembly (e.g., to the first major surface 232 of the substrate 230, to the pins 202, 252, to the potting material 206, 256, etc.). In some embodiments, applying 950 the insulating material includes applying 955 (e.g., depositing, spreading, etc.) an adhesive (e.g., the medical adhesive 840) to the substrate and, more particularly, to the fluid control bed, to bond the insulating material to the feedthrough assembly.

Forming 920 the fluid control bed may include any suitable techniques. Suitable fluid control bed forming techniques may include one or more of the techniques described herein for forming 720 the at least first and second fluid control beds. Forming 925 the bonding surface texture may likewise include any suitable texture-forming techniques, such as one or more of the techniques described herein for forming 720 the at least first and second fluid control beds. In at least one embodiment, forming 925 the bonding surface texture includes femtosecond laser machining the first major surface.

Forming 925 the bonding textures using femtosecond laser machining may advantageously form multi-tiered structures in the substrate surface, such as three-tiered structures including macro-, micro-, and nano-structures. Such multi-tiered structures may advantageously provide increased surface area, tortuosity, and interlock mechanisms, which may, in turn, improve bonding efficacy between the textured substrate surface and an adhesive (e.g., the medical adhesive 840).

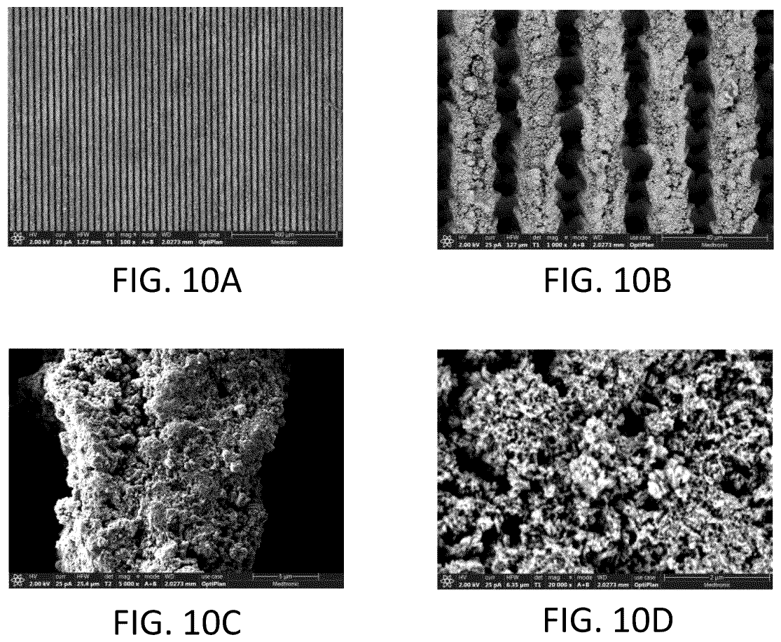

Multi-tiered structures formed by femtosecond laser machining to a depth of approximately 30 micrometers (um) are shown in FIGS. 10A-17D. FIGS. 10A-10D show scanning electron microscopy (SEM) images of one-pass line-textured grade 2 titanium coupons with multi-tiered structures including macro-scale line structures formed in grade 2 titanium coupons by 1 pass of a femtosecond laser at 100× magnification (FIG. 10A), 1,000× magnification (FIG. 10B), 5,000× magnification (FIG. 10C), and 20,000× magnification (FIG. 10D).

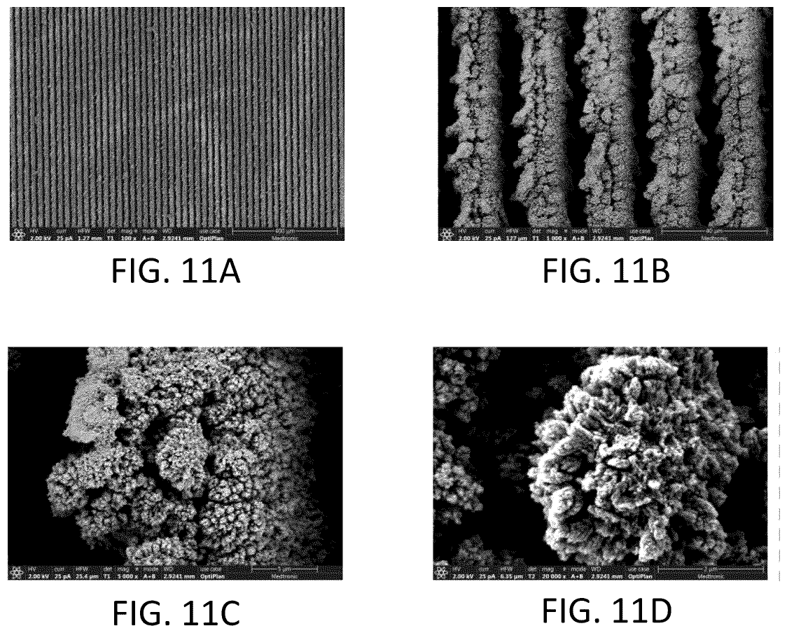

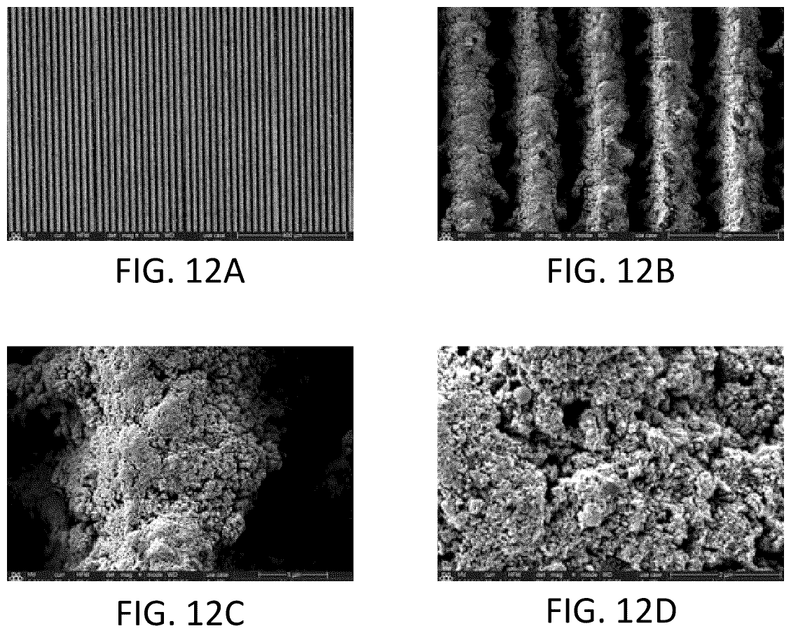

FIGS. 11A-11D show SEM images of two-pass line-textured grade 2 titanium coupons with multi-tiered structures including macro-scale line structures formed in grade 2 titanium coupons by 2 passes of a femtosecond laser at 100× magnification (FIG. 11A), 1,000× magnification (FIG. 11B), 5,000× magnification (FIG. 11C), and 20,000× magnification (FIG. 11D).

FIGS. 12A-12D show SEM images of five-pass line-textured grade 2 titanium coupons with multi-tiered structures including macro-scale line structures formed in grade 2 titanium coupons by 5 passes of a femtosecond laser at 100× magnification (FIG. 12A), 1,000× magnification (FIG. 12B), 5,000× magnification (FIG. 12C), and 20,000× magnification (FIG. 12D).

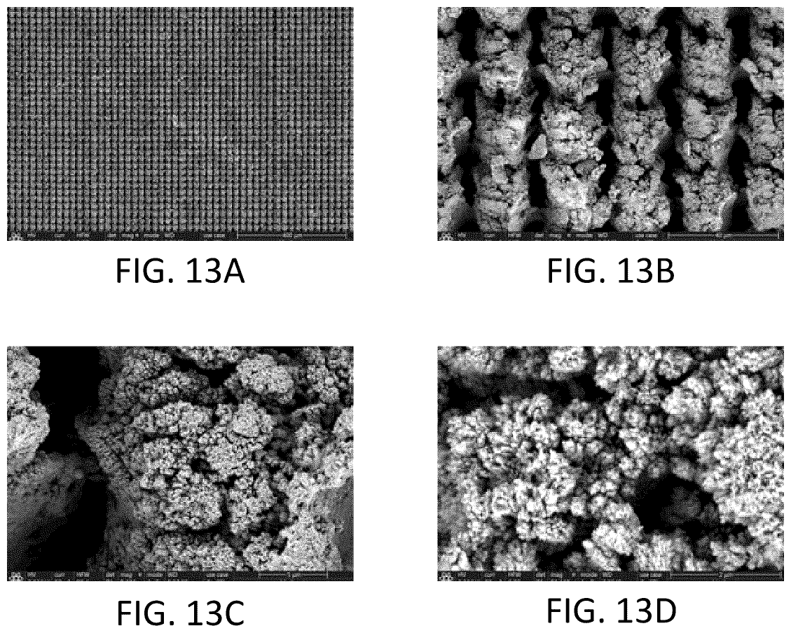

FIGS. 13A-13D show SEM images of one-pass grid-textured grade 2 titanium coupons with multi-tiered structures including macro-scale grid structures formed in grade 2 titanium coupons by 1 pass of a femtosecond laser at 100× magnification (FIG. 13A), 1,000× magnification (FIG. 13B), 5,000× magnification (FIG. 13C), and 20,000× magnification (FIG. 13D).

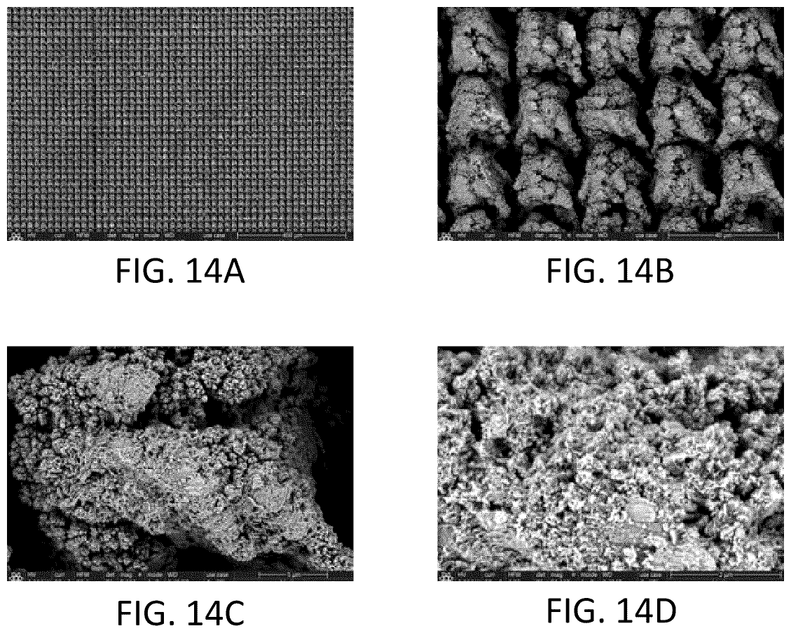

FIGS. 14A-14D show SEM images of two-pass grid-textured grade 2 titanium coupons with multi-tiered structures including macro-scale grid structures formed in grade 2 titanium coupons by 2 passes of a femtosecond laser at 100× magnification (FIG. 14A), 1,000× magnification (FIG. 14B), 5,000× magnification (FIG. 14C), and 20,000× magnification (FIG. 14D).

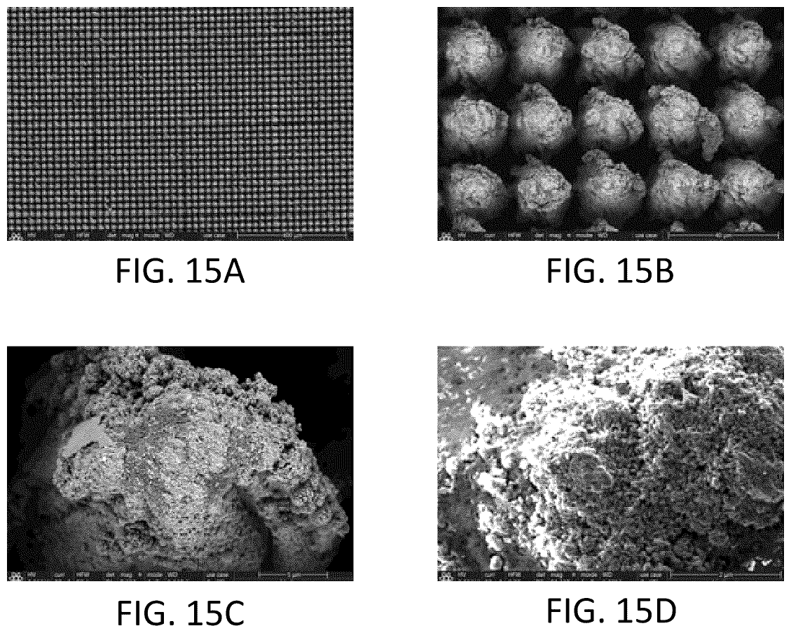

FIGS. 15A-15D show SEM images of five-pass grid-textured grade 2 titanium coupons with multi-tiered structures including macro-scale grid structures formed in grade 2 titanium coupons by 5 passes of a femtosecond laser at 100× magnification (FIG. 15A), 1,000× magnification (FIG. 15B), 5,000× magnification (FIG. 15C), and 20,000× magnification (FIG. 15D).

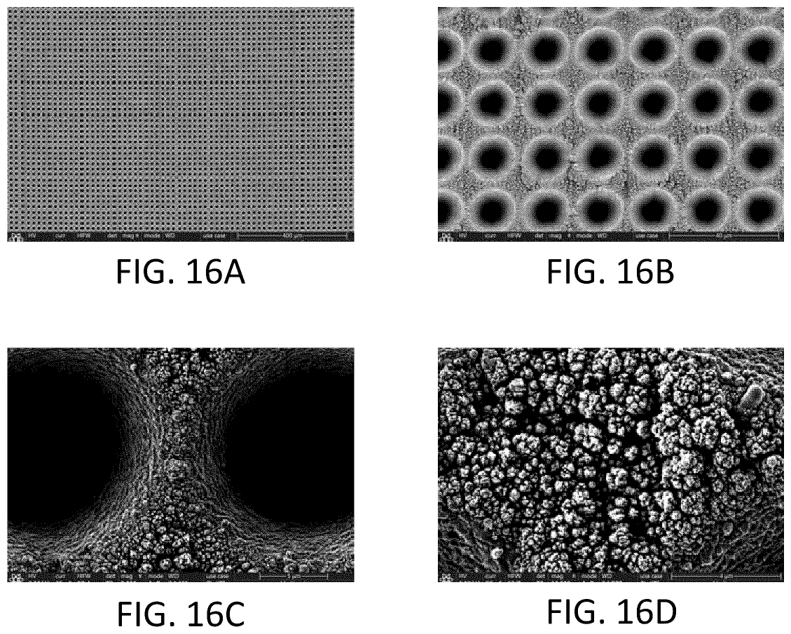

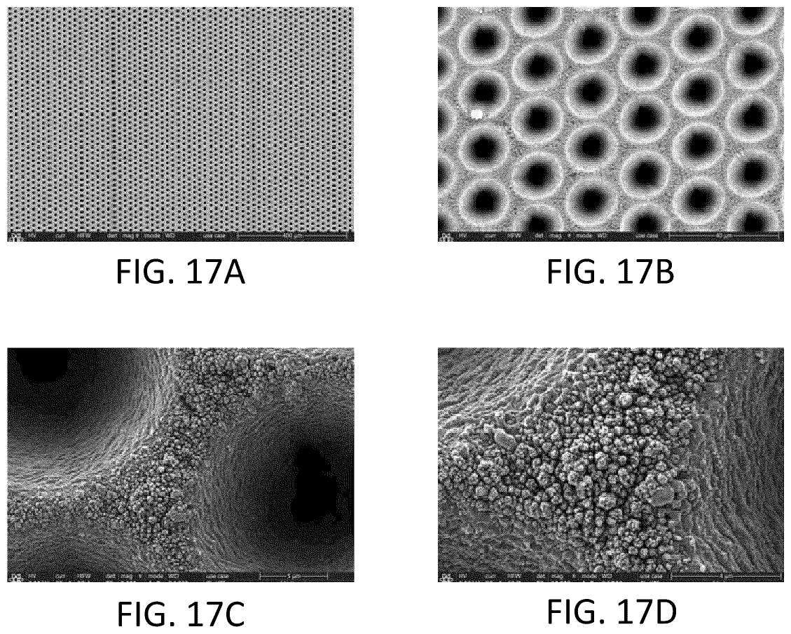

FIGS. 16A-16D show SEM images of dimple-textured grade 2 titanium coupons with multi-tiered structures including macro-scale dimples spaced 20 um (on centers) by a femtosecond laser at 100× magnification (FIG. 16A), 1,000× magnification (FIG. 16B), 5,000× magnification (FIG. 16C), and 10,000× magnification (FIG. 16D).

FIGS. 17A-17D show SEM images of dimple-textured (staggered) grade 2 titanium coupons with multi-tiered structures including macro-scale dimples spaced 20 um (staggered) by a femtosecond laser at 100× magnification (FIG. 17A), 1,000× magnification (FIG. 17B), 5,000× magnification (FIG. 17C), and 10,000× magnification (FIG. 17D).

### ILLUSTRATIVE ASPECTS

The following is a list of illustrative embodiments according to the present disclosure.

Aspect 1 is a substrate for a feedthrough assembly for an implantable medical device, the substrate comprising:
a first major surface and an opposing second major surface;
at least first and second feedthrough cavities defined by the substrate and each extending through the substrate from the first major surface to the second major surface, wherein each feedthrough cavity comprises a respective feedthrough bore configured to receive a feedthrough pin; and
at least first and second fluid control beds on the first major surface, the first fluid control bed forming a first area about the first feedthrough bore configured to preferentially contain, to the first fluid control bed, potting material applied to the first feedthrough bore, the second fluid control bed forming a second area about the second feedthrough bore configured to preferentially contain, to the second fluid control bed, potting material applied to the second feedthrough bore, wherein the first and second fluid control beds are separated from one another.

Aspect 2 is the substrate of aspect 1, wherein the first fluid control bed defines:
a fluid control channel at least partially surrounding the first feedthrough bore and forming part of the first feedthrough cavity, and optionally further defines an outer channel at least partially surrounding the fluid control channel.

Aspect 3 is the substrate of any one of aspects 1-2, wherein the first fluid control bed comprises a bed fluid control surface texture defining a plurality of grooves, wherein the bed fluid control surface texture is configured to preferentially wick a potting material compared to other portions of the first major surface.

Aspect 4 is the substrate of aspect 3, wherein the plurality of grooves comprises a plurality of concentric grooves at least partially surrounding the first feedthrough bore.

Aspect 5 is the substrate of any one of aspects 3-4, wherein the plurality of grooves comprises a plurality of axial grooves substantially normal to and at least partially surrounding the first feedthrough bore.

Aspect 6 is the substrate of any one of aspects 3-5, wherein the plurality of grooves comprises a first plurality of substantially parallel grooves and a second plurality of substantially parallel grooves substantially perpendicular to the first plurality of substantially parallel grooves.

Aspect 7 is the substrate of any one of aspect 3-6, wherein each groove of the plurality of grooves independently has a depth of between 10 micrometers (um) and 20 um, between 5 um and 40 um, or 15 um.

Aspect 8 is the substrate of any one of aspect 3-7, wherein the plurality of grooves has a minimum-to-minimum period of between 10 um and 120 um, between 20 um and 100 um, or 20 um.

Aspect 9 is the substrate of any one of aspects 3-8, wherein the plurality of grooves has a groove aspect ratio of between 1:1 and 1:5, between 1:2 and 1:4, or 1:2.

Aspect 10 is the substrate of any one of aspects 1-9, wherein the first fluid control bed has a depth measured from the major surface of between 10 um and 20 um, between 5 um and 40 um, or 15 um.

Aspect 11 is a feedthrough assembly comprising:
the substrate of any one of aspects 1-10; and
a first feedthrough pin disposed within the first feedthrough bore and configured to electrically connect a component in an internal volume of the implantable medical device with a component in communication with an environment external to the implantable medical device.

Aspect 12 is the feedthrough assembly of aspect 11, wherein the first feedthrough pin comprises a pin fluid control exterior surface texture, the pin fluid control surface exterior texture comprising a plurality of axial grooves to draw the potting material from the first feedthrough bore, the plurality of axial grooves disposed radially about the pin and extending axially along the pin away from the first feedthrough bore,
wherein the pin fluid control exterior surface texture optionally further comprises a stop abutting the plurality of axial grooves and comprising at least one radial groove.

Aspect 13 is an implantable medical device comprising:
a housing defining an internal volume; and
a feedthrough assembly of any one of aspects 11 or 12, wherein the feedthrough assembly is fixed relative to the housing.

Aspect 14 is a method for forming an implantable medical device feedthrough assembly, the method comprising:
providing a substrate having a first major surface and an opposing second major surface, and defining at least first and second feedthrough cavities each extending through the substrate from the first major surface to the second major surface, wherein each feedthrough cavity comprises a respective feedthrough bore;
forming at least first and second fluid control beds on the first major surface, the first fluid control bed forming a first area about the first feedthrough bore configured to preferentially contain, to the first fluid control bed, potting material applied to the first feedthrough bore, the second fluid control bed forming a second area about the second feedthrough bore configured to preferentially contain, to the second fluid control bed, potting material applied to the second feedthrough bore; and
bonding at least first and second feedthrough pins within the respective first and second feedthrough bores using a potting material.

Aspect 15 is the method of aspect 14, wherein the forming at least first and second fluid control beds on the first major surface comprises forming a fluid control channel abutting and at least partially surrounding the first feedthrough bore, and wherein the fluid control bed optionally defines an outer channel abutting and at least partially surrounding the fluid control channel.

Aspect 16 is the method of any one of aspects 14-15, wherein the forming at least first and second fluid control beds on the first major surface comprises forming a bed fluid control surface texture comprising a plurality of grooves.

Aspect 17 is the method of any one of aspects 14-16, wherein the forming at least first and second fluid control beds on the first major surface comprises bead blasting the first major surface, laser etching the first major surface, laser spot-welding the first major surface, machining the first major surface, micro-etching the first major surface, or a combination of two or more thereof.

Aspect 18 is a substrate for a feedthrough assembly for an implantable medical device, the substrate comprising:
a first major surface and an opposing second major surface;
a feedthrough cavity defined by the substrate and extending through the substrate from the first major surface to the second major surface, wherein the feedthrough cavity comprises a respective feedthrough bore configured to receive a feedthrough pin; and
a fluid control bed comprising a bonding surface texture on the first major surface, the fluid control bed defining a bed surface area and a bonding surface area that is at least 2 times the bed surface area.

Aspect 19 is the substrate of aspect 18, wherein the bonding surface texture defines a plurality of grooves, and wherein two or more of the plurality of grooves are optionally concentric.

Aspect 20 is the substrate of aspect 19, wherein each groove of the plurality of grooves independently has a depth of between 10 micrometers (um) and 20 um, between 5 um and 40 um, or 15 um, and optionally wherein the plurality of grooves has a minimum-to-minimum period of between 10 um and 120 um, between 20 um and 100 um, or 20 um.

Aspect 21 is the substrate of any one of aspects 19-20, wherein the plurality of grooves has a groove aspect ratio of between 1:1 and 1:5, between 1:2 and 1:4, or 1:2.

Aspect 22 is the substrate of any one of aspects 18-21, wherein the bonding surface texture defines macro-scale structures, micro-scale structures, nano-scale structures, or any combination thereof; optionally wherein the macro-scale structures comprise grooves, lines, grids, dimples, or any combination thereof.

Aspect 23 is the substrate of any one of aspects 18-21, wherein the bonding surface texture comprises grooves, lines, grids, dimples, or any combination thereof.

Aspect 24 is a method for forming an implantable medical device feedthrough assembly, the method comprising:
providing a substrate having a first major surface and an opposing second major surface, and defining a feedthrough cavity extending through the substrate from the first major surface to the second major surface, wherein the feedthrough cavity comprises a feedthrough bore;
forming a fluid control bed comprising a bonding surface texture on the first major surface, the fluid control bed defining a bed surface area and a bonding surface area that is at least 2 times the bed surface area; and
bonding a feedthrough pin within the feedthrough bore using a potting material.

Aspect 25 is the method of aspect 24, wherein forming the fluid control bed comprising the bonding surface texture on the first major surface comprises forming, using a femtosecond laser, the bonding surface texture on the first major surface.

Aspect 26 is the method of any one of aspects 24-25, wherein the bonding surface texture defines a plurality of grooves, and wherein two or more of the plurality of grooves are optionally concentric.

Aspect 27 is the method of aspect 26, wherein each groove of the plurality of grooves independently has a depth of between 10 micrometers (um) and 20 um, between 5 um and 40 um, or 15 um, and optionally wherein the plurality of grooves has a minimum-to-minimum period of between 10 um and 120 um, between 20 um and 100 um, or 20 um.

Aspect 28 is the method of any one of aspects 26-27, wherein the plurality of grooves has a groove aspect ratio of between 1:1 and 1:5, between 1:2 and 1:4, or 1:2.

Aspect 29 is the method of any one of aspects 24-28, wherein the bonding surface texture defines macro-scale structures, micro-scale structures, nano-scale structures, or any combination thereof; optionally wherein the macro-scale structures comprise grooves, lines, grids, dimples, or any combination thereof.

Aspect 30 is the method of any one of aspects 24-29, wherein the bonding surface texture comprises grooves, lines, grids, dimples, or any combination thereof.

Aspect 31 is a substrate for a feedthrough assembly for an implantable medical device, the substrate comprising:
a first major surface and an opposing second major surface;
at least a first feedthrough cavity defined by the substrate and extending through the substrate from the first major surface to the second major surface, wherein each feedthrough cavity comprises a respective feedthrough bore configured to receive respective a feedthrough pin; and
at least a first fluid control bed comprising a surface texture on the first major surface, the fluid control bed defining a bed surface area and a texture surface area that is at least 1.5 times, at least 2 times, at least 3 times, at least 4 times, at least 5 times, at least 6 times, at least 7 times, at least 8 times, at least 9 times, or at least 10 times the bed surface area.

All publications and patent applications in this specification are indicative of the level of ordinary skill in the art to which this technology pertains. All publications and patent applications are herein incorporated by reference to the same extent as if each individual publication or patent application was specifically and individually indicated by reference. In the event that any inconsistency exists between the disclosure of the present application and the disclosure(s) of any document incorporated herein by reference, the disclosure of the present application shall govern.

This application is intended to cover adaptations or variations of the present subject matter. It is to be understood that the above description is intended to be illustrative, and not restrictive, and the claims are not limited to the illustrative embodiments as set forth herein.

What is claimed is:

1. A feedthrough assembly for an implantable medical device, the feedthrough assembly comprising a substrate comprising:
a first major surface and an opposing second major surface;
at least a first feedthrough cavity defined by the substrate and extending through the substrate from the first major surface to the second major surface, wherein each feedthrough cavity comprises a respective feedthrough bore configured to receive a respective feedthrough pin; and
at least a first fluid control bed comprising a surface texture on the first major surface, the first fluid control bed defining a bed surface area and a texture surface area that is at least 1.5 times the bed surface area.

2. The feedthrough assembly of claim 1, wherein the substrate further comprises:
a second feedthrough cavity defined by the substrate and extending through the substrate from the first major surface to the second major surface, wherein the second feedthrough cavity comprises a respective feedthrough bore configured to receive a respective feedthrough pin; and
a second fluid control bed on the first major surface and separated from the first fluid control bed, wherein the second fluid control bed forms a respective area about the feedthrough bore of the second feedthrough cavity configured to preferentially contain, to the second fluid control bed, potting material applied to the feedthrough bore of the second feedthrough cavity; and
wherein the first fluid control bed forms a respective area about the feedthrough bore of the first feedthrough cavity configured to preferentially contain, to the first fluid control bed, fluid applied to the feedthrough bore of the first feedthrough cavity.

3. The feedthrough assembly of claim 1, wherein the first fluid control bed defines a fluid control channel at least partially surrounding the feedthrough bore of the first feedthrough cavity and forming part of the first feedthrough cavity.

4. The feedthrough assembly of claim 3, wherein the first fluid control bed further defines an outer channel at least partially surrounding the fluid control channel.

5. The feedthrough assembly of claim 1, wherein the surface texture is configured to preferentially wick fluid compared to other portions of the first major surface.

6. The feedthrough assembly of claim 1, wherein the surface texture of the first fluid control bed defines a plurality of grooves, a plurality of lines, a grid, a plurality of dimples, or any combination thereof.

7. The feedthrough assembly of claim 6, wherein the plurality of grooves comprises a plurality of concentric grooves at least partially surrounding the feedthrough bore of the first feedthrough cavity.

8. The feedthrough assembly of claim 6, wherein the plurality of grooves comprises a plurality of axial grooves substantially normal to and at least partially surrounding the feedthrough bore of the first feedthrough cavity.

9. The feedthrough assembly of claim 6, wherein the plurality of grooves comprises a first plurality of substantially parallel grooves and a second plurality of substantially parallel grooves substantially perpendicular to the first plurality of substantially parallel grooves.

10. The feedthrough assembly of claim 6, wherein each groove of the plurality of grooves independently has a depth of between 10 micrometers (um) and 20 um, between 5 um and 40 um, or 15 um.

11. The feedthrough assembly of claim 6, wherein the plurality of grooves has a minimum-to-minimum period of between 10 um and 120 um, between 20 um and 100 um, or 20 um.

12. The feedthrough assembly of claim 6, wherein the plurality of grooves has a groove aspect ratio of between 1:1 and 1:5, between 1:2 and 1:4, or 1:2.

13. The feedthrough assembly of claim 1, wherein the first fluid control bed has a depth measured from the first major surface of between 10 um and 20 um, between 5 um and 40 um, or 15 um.

14. The feedthrough assembly of claim 1, wherein the surface texture defines macro-scale structures, micro-scale structures, nano-scale structures, or any combination thereof.

15. The feedthrough assembly of claim 1, further comprising a feedthrough pin disposed within the feedthrough bore of the first feedthrough cavity and configured to electrically connect a component in an internal volume of the implantable medical device with a component in communication with an environment external to the implantable medical device;
wherein the feedthrough pin comprises a pin fluid control exterior surface texture, the pin fluid control surface exterior texture comprising a plurality of axial grooves to draw fluid from the first feedthrough bore, the plurality of axial grooves disposed radially about the pin and extending axially along the pin away from the feedthrough bore of the first feedthrough cavity.

16. The feedthrough assembly of claim 15, wherein the pin fluid control exterior surface texture further comprises a stop abutting the plurality of axial grooves and comprising at least one radial groove.

17. A method for forming an implantable medical device feedthrough assembly, the method comprising:
providing a substrate having a first major surface and an opposing second major surface, and defining at least a first feedthrough cavity extending through the substrate from the first major surface to the second major surface, wherein each feedthrough cavity comprises a respective feedthrough bore;
forming at least a first fluid control bed comprising a surface texture on the first major surface, the first fluid control bed defining a bed surface area and a texture surface area that is at least 1.5 times the bed surface area;
bonding at least first and second feedthrough pins within the respective first and second feedthrough bores using a potting material.

18. The method of claim 17, wherein the forming at least a first fluid control bed comprises forming a fluid control channel abutting and at least partially surrounding the feedthrough bore of the first feedthrough cavity.

19. The method of claim 18, wherein the first fluid control bed defines an outer channel abutting and at least partially surrounding the fluid control channel.

20. The method of claim 17, wherein the forming at least a first fluid control bed comprises bead blasting the first major surface, laser etching the first major surface, laser spot-welding the first major surface, machining the first major surface, micro-etching the first major surface, or any combination thereof.
